(19) United States
(12) Patent Application Publication
(10) Pub. No.: US 2026/0263720 A1
(43) Pub. Date: Sep. 10, 2026

(54) Wearable Medical Gas Delivery System With Automatic Cord Management

(71) Applicant: Lora Rants, Aurora, CO (US)

(72) Inventor: Lora Rants, Aurora, CO (US)

(21) Appl. No.: 19/558,692

(22) Filed: Mar. 6, 2026

Related U.S. Application Data
(60) Provisional application No. 63/768,798, filed on Mar. 7, 2025.

(51) Int. Cl. A61M 16/06; A61M 16/00

(52) U.S. Cl. CPC A61M 16/0672; A61M 16/0051; A61M 16/0087; A61M 2209/088

(57) ABSTRACT

A wearable medical gas delivery system enhances patient mobility and safety through a body-centric automatic cord management mechanism worn at the patient's center of gravity. The system features a dual-cord configuration wherein a stationary oxygen cord extends to the patient's nasal interface while remaining mechanically isolated from an automatic recoil mechanism managing a fixed hose to the oxygen source. A gas-tight rotary manifold maintains continuous pressurized gas flow during rotation. The biomechanical isolation architecture ensures retraction forces are absorbed by the wearable device body rather than transmitted to the patient's face. A circumferential visual safety indicator positioned one foot from the line's terminal end provides disconnection warning during patient movement.

### CROSS REFERENCE TO RELATED APPLICATIONS

This application claims the benefit of and priority to U.S. Provisional Patent Application No. 63/768,798, filed Mar. 7, 2025, entitled "WEARABLE MEDICAL GAS DELIVERY SYSTEM WITH AUTOMATIC CORD MANAGEMENT," which is incorporated herein by reference in its entirety. BACKGROUND OF THE INVENTION

### BACKGROUND OF THE INVENTION

### Field of the Invention

The present invention relates generally to medical gas delivery systems, and more particularly to wearable oxygen delivery cord management devices that enhance patient mobility while preventing accidental disconnection through automated mechanical recoil mechanisms and integrated visual safety indicators.

### Description of the Related Art

Medical gas delivery systems, particularly supplemental oxygen therapy systems, have relied for decades on a "fixed-source" paradigm wherein patients requiring chronic oxygen therapy navigate their environment using extended lengths of clear PVC tubing connected to stationary oxygen concentrators or wall-mounted medical gas outlets. While standard oxygen line lengths of 25 to 50 feet theoretically provide substantial mobility range, the practical management of these extended cords creates significant challenges that compromise patient safety, limit functional independence, and increase the risk of therapy interruption through accidental disconnection.

The fundamental problem facing oxygen therapy patients is what has been termed the "Cord Management Crisis" wherein excessive slack in oxygen delivery lines creates tangling, tripping hazards, and constant disconnection risk during normal activities of daily living. This problem is particularly acute when patients move closer to their oxygen source, causing substantial cord slack to accumulate on the floor, or when patients extend to maximum range, creating tension that can dislodge nasal cannulas and interrupt essential respiratory therapy.

One category of existing solutions attempts to address cord management through passive coiled tubing systems, exemplified by commercially available products such as Tidy Tubing. These systems utilize the inherent elasticity of spiral-wound polyurethane to provide self-retraction, similar to coiled telephone cords. However, passive coiled systems suffer from a critical mechanical limitation: they exhibit linear increases in tension force as the tubing is extended, with retraction forces often reaching levels of 5 to 10 ounces at full extension. This excessive pulling force is transmitted directly through the oxygen delivery line to the patient's nasal interface, frequently causing nasal cannula displacement, facial discomfort, and interruption of oxygen therapy. Additionally, passive coiled systems are typically limited to 10 to 15 feet of extended length before the coil tension becomes unmanageable, significantly restricting patient mobility compared to standard 25 to 50 foot oxygen lines.

The mechanical tension created by passive coiled systems represents a "pull-on-face" problem wherein the spring force of the coiled tubing acts continuously against the patient's nasal septum and upper lip, creating constant physical strain that patients find uncomfortable and that increases the likelihood of cannula dislodgement during movement. For elderly patients or those with sensitive skin conditions, this continuous pulling force can cause tissue irritation and reduce therapy compliance.

A second category of solutions involves stationary motorized reel systems, including commercial products such as the O2 Remote system and the Buddy retractable oxygen hose reel. These systems position an automated reel mechanism at or near the oxygen source, utilizing motorized retraction systems with electronic sensors and wireless controls to manage oxygen line deployment based on patient distance from the source. While stationary motorized reels effectively manage floor-level slack near the oxygen concentrator, they fail to address the fundamental "dragging distance" problem wherein the patient must still pull the full length of deployed oxygen tubing from the stationary reel location to their nasal interface. This creates a biomechanical "dragging force" wherein the weight and friction of 25 to 50 feet of deployed tubing must be continuously overcome by patient movement, increasing metabolic demand and potentially contributing to oxygen desaturation during ambulation.

Stationary motorized systems suffer from additional limitations including high cost (typically $139 to $400 per unit), electronic complexity requiring battery maintenance or wall power, sensor-based detection systems that introduce response latency delays of 0.5 to 2.0 seconds between patient movement and system response, and physical installation requirements that make them impractical for patients who move between multiple rooms or floors. The "source-side intelligence" architecture of these systems prioritizes keeping tubing organized near the oxygen concentrator rather than optimizing the biomechanical interface at the patient's body, leaving patients to manage the full deployed tubing length during all movements.

Prior art retractable reel systems for medical oxygen, including those disclosed in U.S. Patent Application 2011/0017856 to Penn, have attempted to combine belt-mounting with retractable reel mechanisms. However, these systems employ single continuous oxygen lines wherein the spring-loaded retraction force acts directly on the tubing extending from the reel to the patient's nasal cannula. This architectural limitation creates the same "pull-on-face" problem exhibited by passive coiled systems, as the torsion spring forces generated by the recoil mechanism are transmitted through the oxygen line directly to the patient's facial nasal interface. Penn's system, while featuring a belt clip attachment option, fails to isolate the patient's respiratory interface from the mechanical forces of the retraction mechanism, resulting in constant pulling sensations at the nasal cannula that increase proportionally with the amount of deployed line.

Other prior art retractable oxygen supply mechanisms, including U.S. Patent 6,065,490 to Falcone, focus on stationary mounting configurations designed for attachment to oxygen tanks or generators rather than patient-worn applications. Falcone's system modifies spring-loaded retractable mechanisms originally designed for electrical wire management but maintains the stationary mounting paradigm that requires patients to drag extended tubing lengths during movement. Similarly, U.S. Patent Application 2006/0243282 to Sackman discloses automated oxygen hose reel assemblies featuring mechanical and electrical systems for dispensing or recoiling tubing, but these systems remain fundamentally tethered to stationary mounting points such as wheelchairs or hospital beds rather than providing truly wearable cord management at the patient's center of gravity.

U.S. Patent 5,392,808 to Pierce describes a retractable tubing reel for use with oxygen tanks that maintains the reel mechanism in proximity to the tank rather than the patient, forcing patients to overcome the mechanical resistance and weight of extended tubing throughout their range of motion. This source-side positioning fails to address the biomechanical energy expenditure required for patient movement and does not provide the continuous dynamic cord length optimization that would result from positioning the management system at the patient's body.

Some prior art systems, exemplified by U.S. Patent Application 2016/0060075, employ high-complexity electronic approaches featuring motorized reels with proximity sensors and body-worn transponders to detect patient distance and adjust tubing length automatically. While these systems represent sophisticated engineering solutions, they introduce substantial complexity through their reliance on electronic sensors, computerized controllers, battery power systems, and wireless communication protocols. This electronic complexity creates multiple potential failure modes including battery depletion, sensor malfunction, communication interference, and control system errors, any of which can compromise oxygen delivery reliability. For elderly patients or those with cognitive impairments, the operational complexity of electronic systems with multiple status indicators and control buttons presents usability challenges that reduce therapy compliance.

The electronic architecture of these systems also introduces inherent response latency wherein movement must first be detected by sensors, processed by control algorithms, and then actuated by electric motors, typically requiring 0.5 to 2.0 seconds from movement initiation to cord management response. During this delay period, excess slack accumulates or unwanted tension develops in the oxygen delivery cord, temporarily creating the very hazards the systems are designed to prevent.

Manual spool systems, including U.S. Patent 10,888,682 to Rhyan, require active user engagement to wind or unwind oxygen line through hand-operated cranks or handles. For patients experiencing respiratory distress, physical weakness, or diminished hand dexterity due to age or comorbid conditions, the manual operation requirement renders these systems impractical for continuous use throughout daily activities. Manual systems fail to provide the hands-free, automatic response to movement that is essential for maintaining patient independence during activities such as cooking, personal hygiene, or navigating stairs where both hands must remain free for balance and support.

A fundamental limitation shared across existing oxygen cord management systems is the absence of a dual-cord configuration that mechanically isolates the patient's nasal interface from retraction mechanism forces. Prior art systems employ single continuous oxygen lines extending from the gas source through any intermediate management devices directly to the nasal cannula, creating an unbroken mechanical pathway through which spring forces, motor torques, or elastic tensions are transmitted to the patient's face. This architectural deficiency results in what can be termed "Mechanical Tension Transfer" wherein the dynamic forces required for cord management are experienced by the patient as pulling, tugging, or displacement forces at their respiratory interface.

No prior art system disclosed in the available patent literature provides a wearable hub positioned at the patient's center of gravity featuring a dual-cord architecture wherein a stationary oxygen cord extending from the hub to the patient's nose remains mechanically isolated from a fixed hose connected to an automatic recoil mechanism. This isolation architecture, which would enable all spring retraction forces to be absorbed by the wearable device body and transferred to the patient's belt rather than their face, represents an unmet need in respiratory therapy cord management.

Existing oxygen delivery systems lack proximal visual warning indicators that provide patients with immediate feedback regarding approaching disconnection risk during cord extension. While some medical devices incorporate visual indicators for various status conditions, exemplified by U.S. Patent 8,430,865 to Lair disclosing measurement marks and lengthwise stripes for enteral connection verification, these systems do not address the specific problem of threshold extension warning for oxygen delivery lines during patient movement. Prior art safety indicators, such as those disclosed in U.S. Patent Application 2008/0021381 to Baxter for chemical exposure indication, employ fundamentally different sensing modalities inappropriate for mechanical distance warning.

Existing oxygen therapy safety monitoring relies primarily on distal indicators such as pressure gauges mounted on oxygen tanks or visual displays on oxygen concentrators, requiring patients to direct their attention away from their immediate environment and back toward their oxygen source to assess system status. For patients engaged in activities of daily living, particularly those involving navigation through complex home environments, the cognitive load and physical impracticality of monitoring distant gauge displays while moving renders these safety systems ineffective for preventing accidental disconnection during cord extension.

No prior art system provides an analog visual safety marking positioned directly on the oxygen line at a predetermined distance from the terminal end, providing immediate visual feedback visible within the patient's field of regard during movement. The specific implementation of a high-visibility colored line indicator positioned approximately one foot from the oxygen line's terminal end, creating a "one-step warning" that alerts patients when they are exactly one stride away from maximum safe extension, represents an unaddressed safety enhancement opportunity in oxygen therapy systems.

While retractable leash mechanisms for animal restraint, such as U.S. Patent 6,792,893 to Quintero disclosing retractable leashes with spool assemblies and ratchet mechanisms, provide mechanical precedent for spring-loaded cord management, these systems address fundamentally different engineering requirements. Animal leashes manage only tensile forces along flexible restraint cords, whereas medical gas delivery systems must maintain gas-tight pressure containment while routing pressurized medical gas through rotating joint interfaces. The mechanical requirements for a gas-tight rotary manifold capable of maintaining oxygen flow at 1 to 6 liters per minute (LPM) across 5 to 50 PSI pressure ranges while accommodating continuous 360-degree rotation are not addressed by simple tensile-force management devices designed for animal restraint applications.

The ergonomic and safety requirements for medical devices worn continuously by patients experiencing respiratory compromise differ substantially from those of consumer products designed for intermittent recreational use. Medical oxygen delivery systems require biocompatible materials conforming to ISO 10993 standards, sealed housings preventing contamination of internal components, and fail-safe mechanical designs that ensure therapeutic gas delivery continuity even during mechanism malfunction. These specialized medical requirements are not addressed by animal leash designs optimized for cost, weight, and consumer aesthetics rather than medical-grade reliability and patient safety.

Prior art wearable medical devices, including belt-mounted systems such as those disclosed in U.S. Patent 6,065,154 to Hulings for defibrillator support garments and U.S. Patent 6,908,448 describing devices for transdermal drug delivery, address attachment and positioning requirements for their specific therapeutic modalities but do not optimize housing geometries for the unique requirements of oxygen cord management. Existing wearable medical housings typically employ rectangular or cylindrical form factors dictated by internal component geometry without consideration for anthropometric optimization related to body contour matching, center of gravity positioning, or interference minimization during ambulation and seated activities.

The specific challenges of housing a spring-loaded spool mechanism containing 25 to 50 feet of medical oxygen tubing within a compact form factor optimized for comfortable extended wear at the waist or hip, while maintaining accessible ports for gas-tight oxygen inlet and outlet connections and providing visibility of internal visual indicators, remain inadequately addressed in prior art wearable medical device designs. The optimization of circular versus oblong housing geometries for different belt-wearing positions and patient anthropometries, and the integration of these housing designs with automatic recoil mechanisms and dual-cord architectures, represent unmet needs in wearable oxygen therapy systems.

The prevailing paradigm in oxygen delivery cord management, as evidenced by commercial products such as the Buddy system and O2 Remote, emphasizes "source-side intelligence" wherein automated management occurs at the oxygen concentrator or tank location. This architecture prioritizes keeping tubing organized away from the oxygen source to prevent tangling with furniture or interference with concentrator operation, but fails to optimize the biomechanical interface at the patient's body where movement actually occurs. The result is a system that manages the "last 5 feet" near the oxygen source while requiring patients to manage the "first 45 feet" extending from the source to their body, leaving the majority of the cord management burden on the patient.

The conceptual shift from "source-side" to "body-side" or "patient-centric" cord management, wherein the intelligent response to movement occurs at the patient's center of gravity rather than at a stationary location 25 to 50 feet away, represents a fundamental architectural evolution not taught or suggested by existing prior art. This body-centric approach would enable the patient's movement to directly and immediately actuate cord length adjustment through mechanical coupling at the patient's belt, eliminating the response latencies and "dragging distances" inherent in source-mounted systems while simultaneously isolating the patient's respiratory interface from management mechanism forces.

The existing landscape of oxygen delivery cord management systems suffers from interconnected deficiencies that compromise patient safety, limit functional mobility, and create unacceptable biomechanical interfaces between therapy equipment and patients. Passive systems create excessive facial tension, stationary automated systems fail to eliminate dragging forces, electronic systems introduce complexity and failure modes, manual systems require continuous patient engagement, and no existing system provides the combination of body-worn automatic mechanical recoil with dual-cord biomechanical isolation and proximal visual safety warning. These persistent deficiencies highlight the critical need for innovative solutions that enhance patient mobility and safety while maintaining reliable oxygen delivery through simple, robust mechanical systems positioned at the patient's center of gravity rather than tethered to stationary oxygen sources.

### SUMMARY OF THE INVENTION

The present invention provides a comprehensive medical gas delivery system that addresses key challenges in oxygen therapy through an innovative wearable device design. The system incorporates a standardized medical gas delivery cord system housed within a wearable device body that can be conveniently attached to a belt.

At its core, the invention features a dual-cord configuration comprising a stationary oxygen cord for nose piece placement and a fixed hose connected to an automatic recoil mechanism. This recoil mechanism, similar to a dog leash design, automatically manages cord length by retracting when moving toward the oxygen source and extending when moving away.

The system includes multiple safety features, most notably a visual indicator system with a red line positioned approximately one foot from the oxygen line's end to prevent accidental disconnection. The device offers versatile attachment options through belt straps and loops, or snapping onto a belt loop, while its housing is available in both circular and oblong disc configurations to accommodate different storage preferences.

The invention supports extended range capabilities, with standard oxygen line lengths of up to 25 feet and the flexibility to connect additional line beyond this length or utilize a larger device supporting up to 50 feet of flow. The entire assembly is contained within a lightweight plastic casing, optimizing portability while maintaining functionality.

### BRIEF DESCRIPTION OF THE FIGURES

FIG. 1a illustrates an exemplary view of the wearable medical gas delivery system incorporating the standardized cord system and automatic recoil mechanism with an oxygen tank supply in accordance with an embodiment.

FIG. 1b illustrates a view of the wearable medical gas delivery system in an oblong disc housing configuration, incorporating the standardized cord system and automatic recoil mechanism in connection with an alternative oxygen supply source in accordance with an embodiment.

FIG. 2 shows the dual-cord configuration featuring the stationary oxygen cord for nose piece placement and the fixed hose connected to the retracting mechanism in accordance with an embodiment..

FIG. 3a depicts the automatic recoil mechanism including the spring-loaded spool assembly, central rotating drum, torsion spring assembly, and ratchet and pawl system in accordance with an embodiment in a front view.

FIG. 3b depicts the automatic recoil mechanism including the spring-loaded spool assembly, central rotating drum, torsion spring assembly, and ratchet and pawl system in accordance with an embodiment in an offset view.

FIG. 4a illustrates the ratchet and pawl system integrated within the wearable device body, showing the mechanism that enables controlled extension and retraction of the oxygen line by allowing smooth extension when pulling the line away from the device while preventing unwanted retraction during use in accordance with an exemplary embodiment.

FIG. 4b depicts the torsion spring assembly housed within the wearable device body, demonstrating the component that provides the necessary retraction force for the automatic recoil mechanism while maintaining consistent tension throughout the extension and retraction cycle in accordance with an exemplary embodiment.

FIG. 4c shows the spring-loaded spool assembly within the wearable device body, illustrating the complete assembly that works in conjunction with the torsion spring to provide automated management of oxygen line length during patient movement in accordance with an exemplary embodiment.

FIG. 4d presents the central rotating drum within the wearable device body showing the rotating drum or spool around which the oxygen hose is wound and featuring the dimensional parameters that accommodate proper line management within the device housing in accordance with an exemplary embodiment.

FIG. 5a illustrates the circular housing configuration of the wearable device body designed for coiled oxygen line storage, showing the stationary oxygen cord for nose piece placement, the dual-cord system, and the visual safety indicator system featuring the distinctive red line marking positioned approximately one foot from the end of the oxygen line.

FIG. 5b: Depicts the oblong disc housing configuration of the wearable device body providing an alternative form factor, showing the stationary oxygen cord for nose piece placement, the dual-cord system, and the red line safety indicator positioned approximately one foot from the oxygen line's end, in accordance with an exemplary embodiment.

FIG. 6 shows the multiple attachment options including the belt strap connection and belt loop attachment methods for securing the device to the user.

### DETAILED DESCRIPTION

In a preferred embodiment of the invention, a standardized medical gas delivery cord system provides enhanced compatibility and reliability for oxygen delivery applications. The system comprises a wearable device that, in one embodiment, incorporates a body configured to be worn on a belt, or snapped onto a belt loop, to effectively contain and manage the oxygen hose.

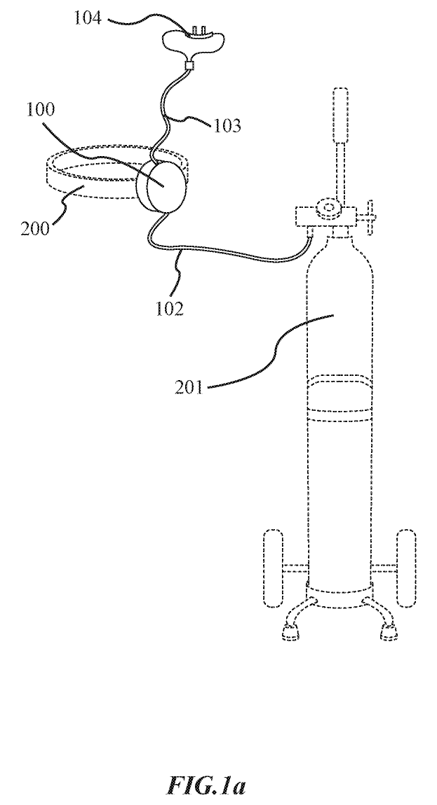

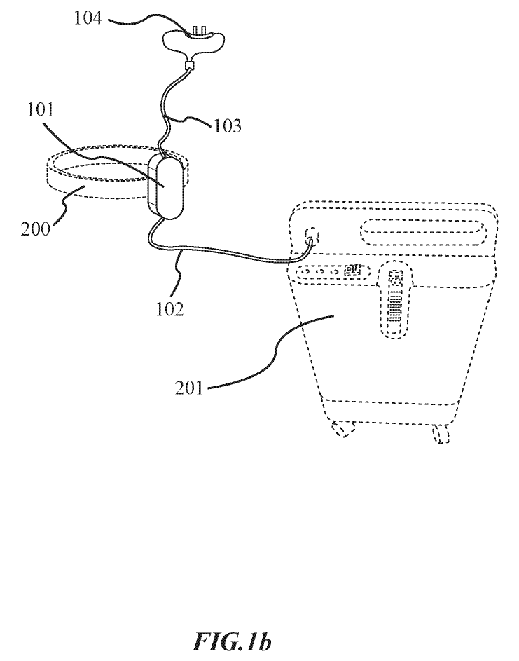

Referring now to the figures, FIG. 1a and FIG. 1b illustrate different views of the wearable medical gas delivery system that incorporates the standardized medical gas delivery cord system contained within the wearable device body. These figures show how the device, though varying in scale and size in various embodiments from those shown in the figures, is configured to be worn on a belt while containing and managing the oxygen hose delivery system.

Referring to FIG. 1a and FIG. 1b, the wearable medical gas delivery system comprises a wearable device body configured to be worn on a belt (200) and connected to an oxygen source (201). The system incorporates the standardized medical gas delivery cord system featuring the dual-cord configuration with a stationary oxygen cord for nose piece placement (104) and fixed hose components (102, 103) that integrate with the retracting mechanism.

In FIG. 1a, the wearable device body is shown in its circular housing configuration (100), while FIG. 1b illustrates the wearable device body in the oblong disc housing configuration (101). Both configurations incorporate the same internal standardized medical gas delivery cord system and automatic recoil mechanism, differing primarily in the external housing form factor to accommodate different user preferences and wearing positions.

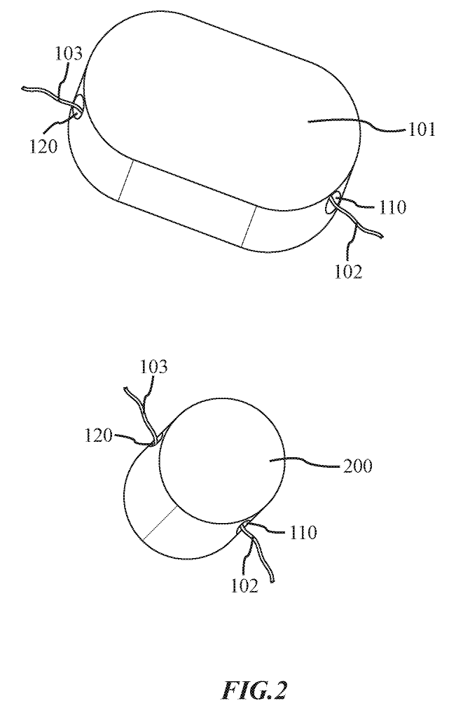

FIG. 2 provides a detailed view of the dual-cord configuration, specifically showing the stationary oxygen cord dedicated to nose piece placement and the fixed hose that extends from the retracting mechanism. This figure demonstrates how these two components work together to provide efficient oxygen delivery while maintaining proper cord management.

In accordance with an embodiment of the invention, a rotary fitting integrated within the wearable device housing enables oxygen to flow continuously through the rotating spool mechanism while maintaining gas-tight sealing during 360-degree rotation operations. The rotary fitting, in one embodiment, comprises a precision-engineered rotary union that couples a stationary gas inlet connected to the oxygen source with a rotating gas outlet connected to the oxygen hose wound on the spool, enabling uninterrupted oxygen flow throughout extension and retraction cycles. This rotary coupling architecture represents a critical differentiating feature that distinguishes medical gas delivery applications from simple tensile-cord management devices such as animal leashes or industrial cable reels, as it must maintain pressure containment and flow continuity during continuous rotation operations.

In accordance with an embodiment of the invention, the rotary fitting comprises a central stationary shaft featuring an internal gas passage aligned with the spool's rotation axis, and a surrounding rotating collar featuring a gas outlet port that rotates with the spool assembly. The interface between stationary shaft and rotating collar incorporates precision bearing surfaces that maintain concentric alignment while accommodating rotational motion, and gas-tight sealing elements positioned at the bearing interface to prevent oxygen leakage during rotation. The bearing surfaces, in one embodiment, comprise self-lubricating bronze bushings, PTFE-lined bearings, or precision ball bearing assemblies that provide smooth rotation with minimal friction throughout the device's operational life.

In accordance with an embodiment of the invention, the gas-tight sealing is achieved through medical-grade elastomeric O-rings positioned in precision-machined grooves at the rotating interface. The O-rings, comprising materials such as nitrile rubber (NBR), ethylene propylene diene monomer (EPDM), or fluoroelastomer (FKM), are selected for oxygen compatibility, resistance to degradation from medical gas exposure, and ability to maintain seal integrity through millions of rotation cycles during the device's service life. The sealing system maintains gas-tight integrity across medical oxygen delivery pressure ranges of 5 to 50 PSI while accommodating continuous bidirectional rotation as the spool extends and retracts the oxygen line.

In accordance with an embodiment of the invention, the rotary fitting maintains consistent oxygen delivery throughout the clinical flow rate range of 1 to 6 liters per minute (LPM) regardless of spool rotation speed or direction. The internal gas passages within the rotary fitting are dimensioned to minimize flow restriction, typically featuring internal diameters of 4mm to 6mm that match or exceed the oxygen hose internal diameter. This flow-matched design ensures the rotary fitting does not create a bottleneck restricting oxygen delivery, maintaining pressure drop across the fitting below 0.2 PSI even at maximum flow conditions. The gas-tight rotary manifold functionality enables the medical gas delivery system to provide reliable therapeutic oxygen flow while simultaneously managing cord length through the rotating spool mechanism, addressing the unique challenge of combining pressurized gas containment with mechanical rotation in a wearable medical device configuration.

As shown in FIG. 2, the dual-cord configuration demonstrates the interaction between the stationary oxygen cord for nose piece placement and fixed hose connected to the retracting mechanism (102, 103), with integrated flow management ports (110, 120) that maintain consistent oxygen delivery during operation.

In accordance with an embodiment of the invention, a gas-tight rotary manifold is integrated within the wearable device housing to enable continuous oxygen flow through the rotating spool mechanism while maintaining pressure integrity during 360-degree rotation operations. The rotary manifold, in one embodiment, comprises a central stationary gas passage aligned with the spool's rotation axis and a rotating gas outlet coupled to the spool assembly, with a precision bearing interface between the stationary and rotating elements that maintains gas-tight sealing during rotation. This rotary union architecture enables the oxygen line wound on the rotating spool to receive continuous gas flow from the stationary oxygen source connection, eliminating the gas flow interruptions or hose tangling that would occur without rotational gas coupling.

In accordance with an embodiment of the invention, the rotary manifold incorporates medical-grade O-ring seals positioned in precision-machined grooves at the interface between stationary and rotating gas passages. The O-ring seals, in one embodiment, comprise medical-grade nitrile rubber (NBR), ethylene propylene diene monomer (EPDM), or fluoroelastomer (FKM) materials selected for compatibility with medical oxygen service and resistance to degradation from ozone exposure. The sealing system maintains gas-tight integrity across the full operating pressure range of medical oxygen delivery systems, typically 5 to 50 PSI, while accommodating continuous rotational movement of the spool assembly without seal binding or excessive friction that would impede recoil mechanism operation.

In accordance with an embodiment of the invention, the rotary manifold's bearing surfaces comprise low-friction materials such as bronze bushings, PTFE bearing surfaces, or ball bearing assemblies that minimize rotational friction while maintaining precise alignment between the stationary and rotating gas passages. The bearing system, in one embodiment, supports radial and axial loads generated by the torsion spring forces and oxygen line tension while maintaining concentric alignment necessary for O-ring seal effectiveness. The combined rotary bearing and gas seal system enables the spool to rotate freely during extension and retraction operations while maintaining continuous oxygen flow, distinguishing the medical gas delivery application from simple tensile-force management devices such as pet leashes or cable reels that do not require pressurized gas containment during rotation.

In accordance with an embodiment of the invention, the rotary manifold maintains consistent oxygen flow rates throughout the clinical delivery range of 1 to 6 liters per minute (LPM) across the full extension range from 0 to 50 feet. The gas passage dimensions within the rotary manifold, typically 4mm to 6mm internal diameter, are sized to minimize flow restriction while maintaining compact dimensions compatible with the wearable device housing. The rotary coupling system maintains flow rate consistency within ±5% across the operating range, ensuring stable therapeutic oxygen delivery regardless of extension length or rotation position.

In accordance with an embodiment of the invention, the integrated flow management ports comprise gas-tight connector assemblies positioned at the oxygen inlet and outlet locations of the wearable device housing. The oxygen inlet port, designated as reference number 110 in the figures, provides connection to the oxygen source supply line and incorporates medical-grade gas fittings conforming to ISO 5356-1 standards for oxygen delivery equipment. The oxygen outlet port, designated as reference number 120, provides connection to the patient's nasal cannula through the stationary oxygen cord and incorporates corresponding medical-grade fittings ensuring universal compatibility with standard respiratory therapy equipment.

In accordance with an embodiment of the invention, the inlet port 110 incorporates threaded fittings, luer-lock connections, or push-to-connect quick-disconnect couplings that provide secure attachment to standard oxygen supply tubing while maintaining gas-tight seals resistant to accidental disconnection. The port assembly, in one embodiment, features a swivel connector that allows the inlet supply line to rotate relative to the device housing, preventing twist accumulation in the supply line as patients rotate or change orientation relative to the oxygen source. The inlet port maintains gas flow integrity across pressure ranges typical of medical oxygen delivery systems, typically 5 to 50 PSI, without leakage or seal degradation throughout the device's service life.

In accordance with an embodiment of the invention, the outlet port 120 connects to the stationary oxygen cord for nose piece placement through a gas-tight connection that maintains stable oxygen flow rates throughout the clinical delivery range. The outlet port, in one embodiment, incorporates flow straightening features or baffles that reduce turbulence and pressure drop, ensuring oxygen delivery rates remain consistent at prescribed levels ranging from 1 to 6 LPM regardless of extension length. The port design minimizes dead space volume within the gas pathway, reducing oxygen waste and ensuring responsive delivery of flow rate changes when patients or clinicians adjust oxygen concentrator settings.

In accordance with an embodiment of the invention, pressure maintenance specifications ensure oxygen flow consistency across the full 0 to 50 foot extension range, with pressure drop not exceeding 0.5 PSI between inlet port 110 and outlet port 120 under maximum flow conditions of 6 LPM. The internal gas pathway through the rotary manifold, ports, and oxygen line is dimensioned to maintain this low pressure drop characteristic, ensuring therapeutic oxygen delivery remains consistent regardless of extension length. Flow rate consistency specifications, in one embodiment, maintain oxygen delivery within ±0.2 LPM of the set flow rate across all extension lengths, providing stable respiratory support throughout patient movement.

In accordance with an embodiment of the invention, the oxygen inlet port 110 comprises a connector assembly with a nominal bore diameter of 1.1 inches (28mm) to accommodate standard medical oxygen supply tubing while maintaining compact device dimensions. The inlet port dimensional specifications, in one embodiment, provide clearance for tubing outer diameters ranging from 0.25 to 0.375 inches (6-10mm), representing the typical size range of medical oxygen delivery tubing used in home healthcare and clinical environments. The port incorporates a chamfered entry and internal barb or compression fitting that ensures secure connection while accommodating minor dimensional variations in supply tubing from different manufacturers.

In accordance with an embodiment of the invention, the oxygen outlet port 120 comprises a connector assembly with a nominal bore diameter of 3.8 inches (97mm) measured at the housing exterior interface, providing clearance for the stationary oxygen cord to exit the housing without restriction during patient movement. The larger outlet port diameter, in one embodiment, accommodates not only the oxygen cord itself but also provides clearance for the cord's full range of angular motion as patients change position relative to the device. The outlet port incorporates a smooth-edged bushing or grommet that prevents abrasion of the stationary oxygen cord while allowing free movement throughout the patient's range of motion.

In accordance with an embodiment of the invention, dimensional parameters for port sizing are optimized for medical-grade connector compatibility, ensuring the device interfaces properly with standard oxygen delivery equipment including: concentrators providing 5-10 LPM maximum flow; oxygen cylinders with standard CGA 540 or CGA 870 fittings and downstream regulators; and nasal cannula assemblies with standard 7-foot supply tubes. The standardized port dimensions ensure universal compatibility across the diverse range of oxygen delivery equipment encountered in clinical practice, eliminating the need for custom adapters or special-purpose fittings.

In accordance with an embodiment of the invention, the device housing is configured to accommodate both 25-foot and 50-foot hose configurations within dimensional envelopes optimized for wearable applications. The 25-foot configuration, in one embodiment, utilizes a spool diameter of 3 to 4 inches and housing diameter of 4 to 5 inches, while the 50-foot configuration utilizes a spool diameter of 4 to 5 inches and housing diameter of 5 to 6 inches. Both configurations maintain housing depth dimensions of 1 to 2 inches, ensuring the complete device profile remains within comfortable wearable dimensions that do not interfere with patient mobility or create discomfort during extended wear periods.

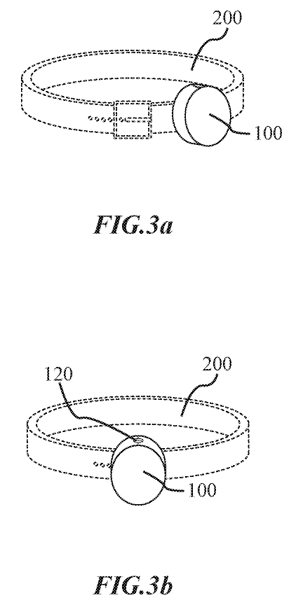

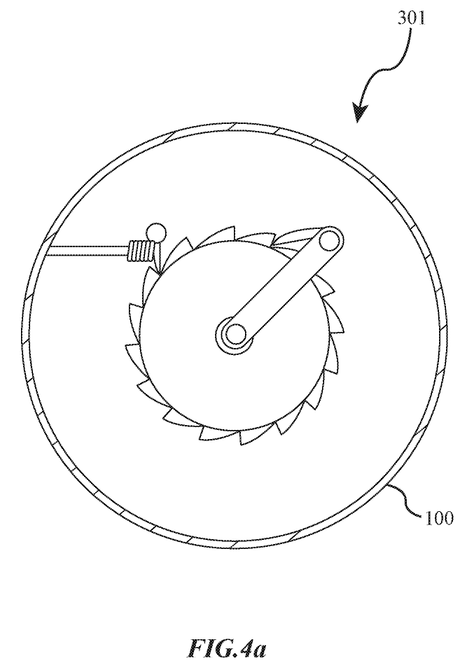

FIG. 3a and FIG. 3b illustrate the automatic recoil mechanism in detail, showing the spring-loaded spool assembly, central rotating drum, torsion spring assembly, and ratchet and pawl system. These components work together to enable automatic retraction when moving toward the oxygen source and controlled extension when moving away, similar to the operation of a dog leash mechanism.

FIG. 3a and FIG. 3b illustrate the wearable device body's integration with the belt attachment system (200), demonstrating how the automatic recoil mechanism is housed within the device while maintaining proper oxygen flow through the integrated port (120).

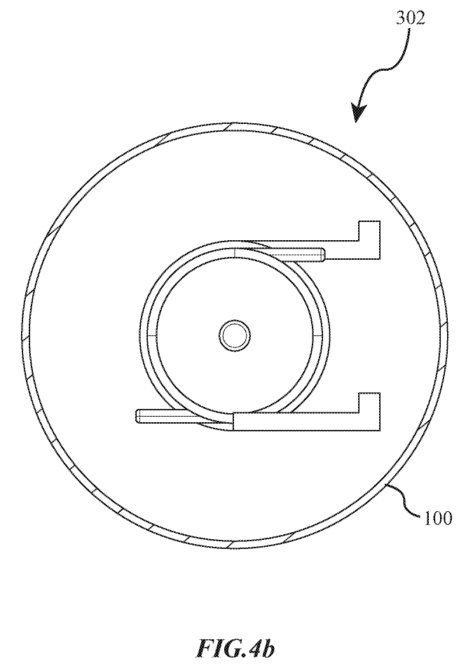

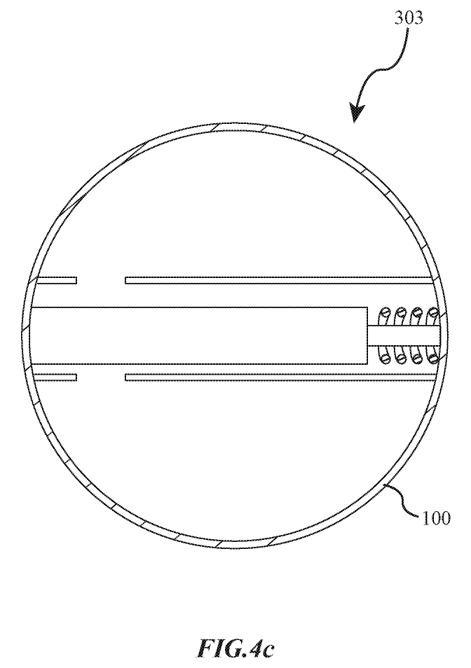

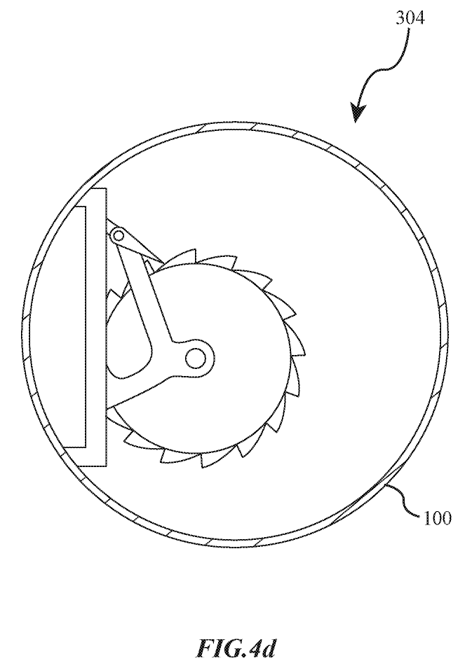

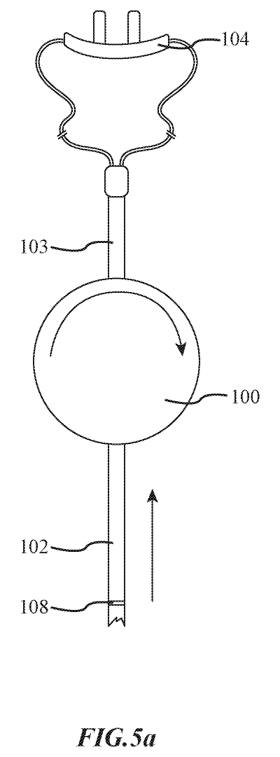

FIG. 4a through FIG. 4d illustrate the individual components of the automatic recoil mechanism housed within the wearable device body (100). FIG. 4a shows the ratchet and pawl system (301), FIG. 4b shows the torsion spring assembly (302), FIG. 4c shows the spring-loaded spool assembly (303), and FIG. 4d shows the central rotating drum (304). These components work together to enable controlled extension and automatic retraction of the oxygen line.

FIGS. 4a through 4d detail the automatic recoil mechanism components. The ratchet and pawl system (301) enables controlled extension and retraction of the oxygen line. The torsion spring assembly (302) provides the necessary retraction force, while working in conjunction with the spring-loaded spool assembly (303). The central rotating drum (304) features specific dimensional parameters (75mm, 50mm, 0mm) to accommodate proper line management within the wearable device body.

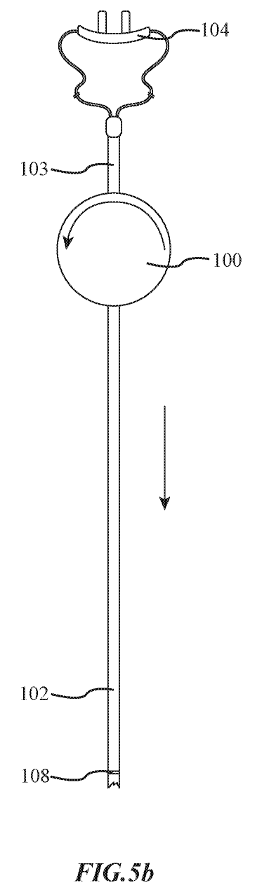

FIG. 5a and FIG. 5b present the two available housing configurations of the wearable device body. FIG. 5a shows the circular shape version specifically designed to accommodate coiled oxygen line storage, while FIG. 5b illustrates the alternative oblong disc configuration. Both configurations are constructed using lightweight plastic casing to optimize portability.

FIGS. 5a and 5b demonstrate the wearable device body configurations in the circular housing (100) and the oblong disc housing (101), respectively, incorporating the nose piece placement component (104), dual-cord system (102, 103), and the visual safety indicator system featuring the distinctive red line marking (108) positioned approximately one foot from the end of the oxygen line.

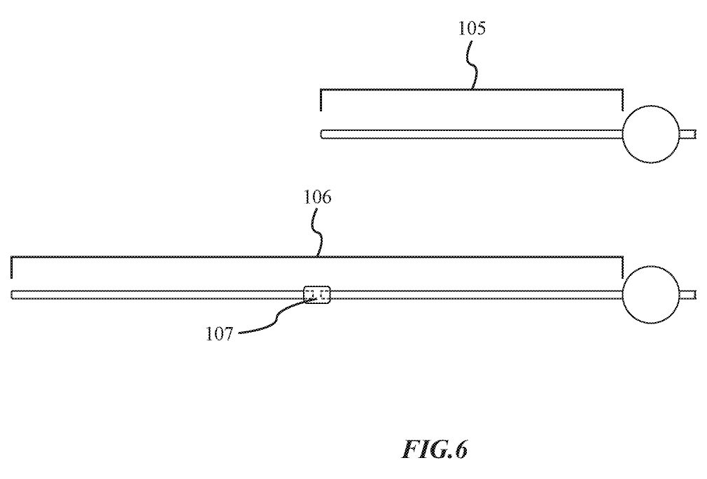

FIG. 6 illustrates the multiple attachment options available for securing the device, including both the belt strap connection and belt loop attachment methods. This figure demonstrates how these different attachment configurations allow users to securely fasten the device according to their preferences while maintaining proper functionality of the oxygen delivery system.

FIG. 6 illustrates the multiple attachment options, showing how the device can be secured through both standard length (105) and extended length (106) configurations, with integrated connection points (107) that enable secure mounting to the user's belt or belt loop.

The standardized medical gas delivery system integrates these components to provide efficient oxygen delivery while maintaining safety through the visual indicator system and automated cord management features. The wearable device’s body in various embodiments is available in both circular and oblong disc configurations to accommodate different user preferences, while maintaining consistent functionality of all integrated components including the automatic recoil mechanism, dual-cord system, and visual safety indicators.

In accordance with an embodiment of the invention, the wearable medical gas delivery system functions as a patient-centric biomechanical hub that represents a fundamental architectural shift from traditional source-side oxygen delivery management systems. Unlike portable oxygen concentrators that replace the stationary oxygen source itself, the present invention retrofits existing stationary oxygen delivery infrastructure by relocating cord management intelligence from the oxygen source location to the patient's body. This body-centric hub architecture enables patients to retain the therapeutic benefits of high-flow stationary oxygen systems while achieving mobility characteristics previously available only with limited-capacity portable units.

In accordance with an embodiment of the invention, the biomechanical hub positioning at the patient's center of gravity creates a dynamic response system that adapts to patient movement in real-time without requiring electronic sensors, battery power, or computerized controls. The mechanical intelligence embedded within the wearable device body responds instantaneously to directional changes, postural shifts, and ambulation patterns, providing continuous cord length optimization throughout activities of daily living. This patient-side intelligence architecture eliminates the limitations inherent in source-side cord management systems where the oxygen source location constrains the geometric efficiency of cord deployment and creates unsafe slack conditions when patients move closer to the source.

In accordance with an embodiment of the invention, the body-centric hub architecture positions the mechanical cord management system at the patient's center of gravity, typically at the waist or hip region when secured to a belt, creating an optimal geometric reference point for omnidirectional movement. This central positioning enables the device to function as a dynamic radius hub wherein the patient can move in any direction relative to the oxygen source while the recoil mechanism automatically adjusts cord deployment from the body outward to the source, rather than from the source outward to the patient as in conventional systems. The body-centric configuration eliminates the geometric inefficiencies and safety hazards created by source-side management systems, particularly the excessive cord slack that accumulates on the floor when patients move toward fixed oxygen sources.

In accordance with an embodiment of the invention, the shift from source-centric to body-centric cord management fundamentally alters the mechanical relationship between the patient's respiratory interface and the recoil mechanism. By positioning the automatic recoil mechanism on the patient's body rather than at the oxygen source, the invention ensures that all retraction forces are absorbed at the patient's center of gravity through the belt attachment system, completely isolating the patient's facial nasal interface from any mechanical pulling forces during cord retraction operations. This biomechanical isolation represents a critical safety improvement over systems where spring-loaded mechanisms positioned at the oxygen source transmit retraction forces directly through the oxygen delivery cord to the patient's nose.

In accordance with an embodiment of the invention, the wearable device functions as a biomechanical tension buffer that absorbs dynamic movement forces at the patient's center of gravity while maintaining stable oxygen delivery to the respiratory interface. The tension buffer architecture, in one embodiment, positions the recoil mechanism between the oxygen source connection point and the patient's facial interface, creating a mechanical isolation zone that prevents forces generated by spring retraction or patient movement from transmitting through to the nasal cannula. This buffer positioning contrasts fundamentally with source-side recoil systems wherein spring forces act directly on the oxygen line extending to the patient's face, potentially disturbing the nasal interface during retraction operations.

In accordance with an embodiment of the invention, the mechanical buffer absorbs force variations through multiple mechanisms including torsion spring compliance, ratchet and pawl energy dissipation, and friction damping at guide roller interfaces. The combined effect of these mechanical damping elements creates a force isolation characteristic wherein rapid patient movements or sudden directional changes generate forces absorbed within the device housing rather than transmitted to the patient's respiratory interface. The stationary oxygen cord extending from the device outlet port 120 to the patient's nose piece remains substantially tension-free throughout normal operation, experiencing only the minimal weight-induced tension of the cord itself rather than dynamic forces from the recoil mechanism.

In accordance with an embodiment of the invention, the biomechanical buffer functionality represents the device's primary differentiating characteristic from simple cord retraction mechanisms, transforming a basic spring-loaded reel into a medical device specifically engineered for respiratory therapy applications. The buffer architecture ensures mechanical cord management operations occur independently from oxygen delivery stability, enabling reliable therapeutic gas flow while simultaneously providing automated cord slack management that enhances mobility and safety.

The automatic recoil mechanism's spring-loaded design, similar to retractable dog leash mechanisms, enables automated management of oxygen line length during patient movement. This system automatically retracts excess line when moving toward the oxygen source while providing smooth extension when moving away, supporting standard lengths up to 25 feet and extended configurations up to 50 feet while maintaining proper oxygen delivery performance.

In the preferred embodiment, the invention utilizes a dual-cord configuration featuring one stationary oxygen cord dedicated to nose piece placement and a fixed hose extending from the retracting mechanism. An example of the invention includes an automatic recoil mechanism, similar in operation to a dog leash, which automatically retracts the oxygen line when moving toward the oxygen source and extends when moving away.

In an embodiment of the invention, a visual safety indicator system incorporates a red line positioned approximately one foot from the end of the oxygen line, serving as a critical disconnection prevention feature. The preferred embodiment includes multiple attachment options, whereby the device can be secured via either a belt strap connection or belt loop attachment.

An example of the invention's extended range capabilities includes a standard oxygen line length of up to 25 feet, with an embodiment that provides the ability to connect additional line beyond the standard length. In another embodiment, a larger device configuration supports oxygen flow for distances up to 50 feet.

In one embodiment, the housing design is available in two configurations: a circular shape version specifically designed for coiled oxygen line storage, and an oblong disc configuration. The preferred embodiment utilizes a lightweight plastic casing to optimize portability.

In a preferred embodiment of the invention, the wearable device comprises a body specifically designed to be worn on a belt, or snapped onto a belt loop, providing a secure and convenient means for containing and managing the oxygen hose system. The preferred embodiment features multiple attachment configurations, including a belt strap connection and a belt loop attachment, allowing users to securely fasten the device according to their preferences.

In one embodiment, the device body is available in two distinct housing configurations. The first configuration features a circular shape specifically designed to accommodate coiled oxygen line storage, while an alternative embodiment utilizes an oblong disc configuration. The preferred embodiment incorporates a lightweight plastic casing design that optimizes portability while maintaining structural integrity. This casing houses the automatic recoil mechanism and provides protection for the contained oxygen line components.

In accordance with an embodiment of the invention, the circular housing configuration is optimized for anterior belt placement at the frontal waist or hip area, where the circular profile distributes the device mass symmetrically around a central axis aligned with the patient's sagittal plane. The circular geometry, typically featuring a diameter of 3 to 5 inches and a depth of 1 to 2 inches, minimizes interference with arm swing during ambulation and reduces the likelihood of contact with fixed environmental objects when the patient moves through doorways or navigates tight spaces. The symmetric weight distribution of the circular design prevents rotational moments that could cause the device to shift position on the belt during dynamic movement.

In accordance with an embodiment of the invention, the oblong housing configuration is optimized for lateral hip placement along the patient's side, where the elongated profile aligns with the body's natural contours and the pelvis's curvature. The oblong design, in one embodiment, features major and minor axis dimensions of approximately 5 inches by 3 inches with a depth of 1 to 1.5 inches, creating a flattened profile that conforms to the hip region while maintaining sufficient internal volume for the spool mechanism. The elongated geometry reduces the anterior-posterior projection distance from the belt, minimizing interference with seated positions and reducing the device's tendency to contact armrests, desk edges, or vehicle seat components during daily activities.

In accordance with an embodiment of the invention, the rear surface of both housing configurations features a contoured profile that matches the body's curvature in the relevant attachment region, enhancing comfort during extended wear periods. The contoured back surface, in one embodiment, comprises a concave profile with a radius of curvature approximating the patient's waist or hip radius, typically ranging from 4 to 8 inches depending on patient anthropometry. This body-conforming design distributes contact pressure across a broader surface area, reducing localized pressure points that could cause discomfort during prolonged wear periods of 8 to 16 hours daily.

In accordance with an embodiment of the invention, the device's center of gravity is positioned to align with the belt attachment points, minimizing rotational moments and maintaining stable positioning during patient movement. The internal component layout, including spool placement, spring assembly location, and housing wall thickness distribution, is engineered to achieve center of gravity alignment that prevents the device from tilting forward or backward relative to the belt plane. Weight distribution analysis, in one embodiment, ensures that the device mass remains balanced within ±0.25 inches of the belt attachment centerline, preventing the torque-induced rotation that would otherwise cause the device to shift position during walking, bending, or transitional movements.

In accordance with an embodiment of the invention, the circular housing configuration is optimized for anterior belt placement at the frontal waist or hip area, where the circular profile distributes the device mass symmetrically around a central axis aligned with the patient's sagittal plane. The circular geometry, typically featuring a diameter of 3 to 5 inches and a depth of 1 to 2 inches, minimizes interference with arm swing during ambulation and reduces the likelihood of contact with fixed environmental objects when the patient moves through doorways or navigates tight spaces. The symmetric weight distribution of the circular design prevents rotational moments that could cause the device to shift position on the belt during dynamic movement.

In accordance with an embodiment of the invention, the oblong housing configuration is optimized for lateral hip placement along the patient's side, where the elongated profile aligns with the body's natural contours and the pelvis's curvature. The oblong design, in one embodiment, features major and minor axis dimensions of approximately 5 inches by 3 inches with a depth of 1 to 1.5 inches, creating a flattened profile that conforms to the hip region while maintaining sufficient internal volume for the spool mechanism. The elongated geometry reduces the anterior-posterior projection distance from the belt, minimizing interference with seated positions and reducing the device's tendency to contact armrests, desk edges, or vehicle seat components during daily activities.

In accordance with an embodiment of the invention, the rear surface of both housing configurations features a contoured profile that matches the body's curvature in the relevant attachment region, enhancing comfort during extended wear periods. The contoured back surface, in one embodiment, comprises a concave profile with a radius of curvature approximating the patient's waist or hip radius, typically ranging from 4 to 8 inches depending on patient anthropometry. This body-conforming design distributes contact pressure across a broader surface area, reducing localized pressure points that could cause discomfort during prolonged wear periods of 8 to 16 hours daily.

In accordance with an embodiment of the invention, the device's center of gravity is positioned to align with the belt attachment points, minimizing rotational moments and maintaining stable positioning during patient movement. The internal component layout, including spool placement, spring assembly location, and housing wall thickness distribution, is engineered to achieve center of gravity alignment that prevents the device from tilting forward or backward relative to the belt plane. Weight distribution analysis, in one embodiment, ensures that the device mass remains balanced within ±0.25 inches of the belt attachment centerline, preventing the torque-induced rotation that would otherwise cause the device to shift position during walking, bending, or transitional movements.

In a preferred embodiment of the invention, the housing comprises a lightweight plastic casing specifically engineered to optimize portability while maintaining structural integrity and durability.

The preferred embodiment incorporates medical-grade plastic materials suitable for healthcare environments, designed to house both the automatic recoil mechanism and the standardized medical gas delivery system components. This casing design is compatible with both the circular shape version for coiled oxygen line storage and the oblong disc configuration. In an alternative embodiment, the housing comprises aluminum as its primary material.

In accordance with an embodiment of the invention, the aluminum housing construction comprises a lightweight metallic alloy specifically selected for institutional healthcare applications where enhanced durability and repeated sterilization are required. The aluminum embodiment provides superior structural rigidity compared to plastic alternatives while maintaining comparable weight characteristics, typically weighing between 4 to 8 ounces for the complete housing assembly. This metallic construction offers improved resistance to impact damage in high-traffic clinical environments and enhanced thermal stability across a wider temperature range of -20°C to 60°C, exceeding the operational range of plastic embodiments.

In accordance with an embodiment of the invention, the aluminum housing features an anodized surface treatment that provides both corrosion resistance and compatibility with common hospital-grade disinfectants including quaternary ammonium compounds, sodium hypochlorite solutions, and alcohol-based sanitizers. The anodized finish, in one embodiment, incorporates antimicrobial properties through the integration of silver ion technology within the surface layer, providing continuous bacterial growth inhibition between cleaning cycles. The aluminum housing embodiment maintains structural integrity through repeated autoclave sterilization cycles at temperatures up to 134°C, making it suitable for shared equipment applications in multi-patient care facilities.

In accordance with an embodiment of the invention, the housing comprises medical-grade acrylonitrile butadiene styrene (ABS) plastic conforming to ISO 10993 biocompatibility standards for prolonged skin contact applications. The ABS formulation, in one embodiment, incorporates impact modifiers and UV stabilizers that provide durability under repeated handling and exposure to ambient lighting conditions in healthcare environments. The material maintains structural integrity across temperature ranges of 0°C to 50°C, ensuring reliable operation in climate-controlled indoor environments typical of hospitals, nursing facilities, and residential settings.

In accordance with an embodiment of the invention, alternative housing embodiments comprise medical-grade polycarbonate plastic that provides enhanced impact resistance and optical clarity compared to ABS alternatives. The polycarbonate formulation offers superior resistance to cracking or fracturing when subjected to drop impacts from waist height, a common occurrence in real-world patient use scenarios. The housing material, in one embodiment, incorporates antimicrobial additives comprising silver ion compounds or copper-based agents that inhibit bacterial colonization on device surfaces, reducing pathogen transmission risks in shared equipment applications or multi-patient healthcare facilities.

In accordance with an embodiment of the invention, medical-grade silicone gaskets and seals are incorporated at housing mating surfaces and port openings to provide environmental protection for internal mechanical components. The silicone gasket material, complying with USP Class VI biocompatibility requirements, prevents moisture intrusion and particulate contamination while maintaining flexibility across the device's operational temperature range. The housing assembly, in one embodiment, achieves an IP42 ingress protection rating, protecting internal components from solid objects greater than 1mm diameter and water droplets from condensation or incidental splashing during cleaning procedures.

In accordance with an embodiment of the invention, the housing materials are selected for compatibility with common healthcare disinfection protocols including quaternary ammonium wipes, 70% isopropyl alcohol solutions, and diluted sodium hypochlorite (bleach) solutions up to 1000 ppm concentration. The plastic materials resist chemical degradation, discoloration, and mechanical property deterioration when subjected to repeated disinfection cycles, typically maintaining structural and aesthetic integrity through 500 or more cleaning procedures over the device's service life. Sterilization compatibility, in one embodiment, extends to low-temperature hydrogen peroxide plasma sterilization for applications requiring terminal sterilization between patient uses in shared equipment scenarios.

In one embodiment, the plastic casing is constructed to accommodate the internal components while minimizing overall weight, making it comfortable for extended wear on a belt or belt loop. The casing design provides sufficient protection for the enclosed recoil mechanism and oxygen line components while maintaining accessibility for necessary maintenance or adjustments.

In accordance with an embodiment of the invention, the two-piece housing construction provides field-replaceable access to the internal oxygen hose without requiring specialized tools or factory service. The housing assembly, in one embodiment, comprises a front housing section and a rear housing section joined through snap-fit latches, threaded fasteners, or quarter-turn locking mechanisms that enable separation of the housing halves for internal component access. This serviceable design allows healthcare facility maintenance personnel or home healthcare providers to replace worn or damaged oxygen hoses, extending device service life and reducing total ownership costs.

In accordance with an embodiment of the invention, the oxygen hose connects to the internal spool mechanism and gas flow manifold through standardized medical gas fittings that facilitate disconnection and reconnection during hose replacement procedures. The hose attachment points, in one embodiment, comprise ISO-standard push-to-connect fittings or threaded luer-lock connectors that provide gas-tight seals while enabling tool-free removal during maintenance operations. The standardized connection interfaces ensure replacement hoses from authorized suppliers maintain proper fit and gas flow characteristics matching original equipment specifications.

In accordance with an embodiment of the invention, the housing interior layout positions the spool assembly and hose routing path for optimal accessibility when the housing is opened, minimizing the complexity of hose replacement procedures. Visual guides or color-coded markings on internal components indicate proper hose routing paths and connection sequences, ensuring correct reassembly following maintenance operations. The field-replaceable design significantly reduces device downtime and eliminates the need for return-to-manufacturer service for routine hose wear replacement, improving availability of oxygen delivery systems in clinical and home healthcare settings.

In accordance with an embodiment of the invention, the housing assembly comprises a two-piece hinged construction featuring an inner housing component and an outer housing component that separate along a parting line to provide comprehensive internal access. The hinged connection, in one embodiment, comprises a living hinge molded integrally with the housing plastic material or a mechanical pin hinge that allows the outer housing to rotate away from the inner housing, exposing the spool assembly, torsion spring, ratchet mechanism, and oxygen hose routing for maintenance, inspection, or component replacement. The hinged architecture enables single-handed opening operations while maintaining permanent connection between housing halves, preventing loss of housing components during service procedures.

In accordance with an embodiment of the invention, the inner housing component provides structural mounting points for the spool's central shaft, torsion spring anchor points, ratchet mechanism mounting, and rotary manifold assembly. The inner housing, in one embodiment, features precision-molded bosses, posts, and bearing surfaces that maintain critical dimensional relationships between rotating and stationary components, ensuring proper mechanical operation and gas-tight sealing throughout the device's service life. The inner housing's structural design provides torsional rigidity that prevents housing deflection under spring loads, maintaining alignment precision necessary for smooth ratchet operation and rotary seal integrity.

In accordance with an embodiment of the invention, the outer housing component provides protective enclosure for the internal mechanisms while incorporating the belt attachment features, port openings, and external cosmetic surfaces. The outer housing, in one embodiment, features recessed areas accepting snap-fit latches, threaded inserts for screw fasteners, or magnetic closure elements that secure the housing halves in the closed position during normal use. The closure mechanism provides sufficient clamping force to maintain housing integrity during patient movement while allowing intentional opening with manual force or simple tool assistance during maintenance procedures.

In accordance with an embodiment of the invention, the two-piece architecture enables efficient manufacturing through separate molding of inner and outer components with subsequent assembly, allows component replacement or upgrade without complete device replacement, and facilitates cleaning of housing interior surfaces to maintain hygiene standards. The snap-fit, screw-together, or magnetically-coupled closure systems enable tool-free or simple-tool opening procedures compatible with field service operations by healthcare facility maintenance personnel, avoiding the need for factory returns for routine maintenance or component replacement.

The preferred embodiment features a casing design that integrates seamlessly with the multiple attachment options, including both belt strap connections and belt loop attachments. This integration ensures secure mounting while maintaining the lightweight characteristics essential for patient mobility.

In an embodiment of the invention, the casing is dimensioned to accommodate standard oxygen line lengths of up to 25 feet when coiled, while alternative configurations support extended lengths up to 50 feet when coiled without compromising the lightweight design objectives. The casing construction ensures proper protection and operation of the visual safety indicator system and recoil mechanism throughout the full range of motion.

In an embodiment of the invention, the wearable body is dimensioned to accommodate standard oxygen line lengths of up to 25 feet. Another embodiment features a larger device configuration capable of managing extended oxygen line lengths of up to 50 feet while maintaining proper functionality of the retracting mechanism. The preferred embodiment integrates the dual-cord system within the wearable body, accommodating both the stationary oxygen cord for nose piece placement and the fixed hose connected to the retracting mechanism. This configuration enables effective management of excess cord length while maintaining consistent oxygen delivery during patient movement.

In accordance with an embodiment of the invention, the wearable device housing accommodates interchangeable spool assemblies of varying capacities, enabling a single housing design to support multiple oxygen line lengths through spool replacement rather than complete device replacement. The modular spool system, in one embodiment, comprises standardized mounting interfaces wherein spool assemblies of 25-foot, 50-foot, or intermediate capacities mount to a common central shaft and drive mechanism within the housing. This interchangeable architecture allows healthcare facilities to maintain a fleet of standardized housing units while customizing capacity to individual patient needs through spool selection.

In accordance with an embodiment of the invention, the standardized spool mounting interface comprises a central keyed shaft that engages with the torsion spring assembly and ratchet mechanism regardless of spool diameter or capacity. Spool assemblies ranging from compact 3-inch diameter spools accommodating 25 feet of oxygen line to larger 5-inch diameter spools accommodating 50 feet of line utilize identical mounting shaft interfaces, ensuring mechanical compatibility across the capacity range. The torsion spring assembly, in one embodiment, features adjustable tension settings that can be calibrated to match the spool capacity, providing appropriate retraction force regardless of the total line length wound on the spool.

In accordance with an embodiment of the invention, the modular capacity system enables healthcare providers to adjust device capacity as patient mobility needs evolve, upgrading from a 25-foot spool to a 50-foot spool without purchasing a completely new device. The spool interchange procedure, in one embodiment, is accomplished by opening the two-piece housing assembly, removing the existing spool from the central mounting shaft, installing the replacement spool of desired capacity, and adjusting the torsion spring tension setting to match the new spool capacity. This modularity reduces equipment costs for healthcare facilities while providing flexibility to accommodate varying patient mobility requirements across different care settings.

In a preferred embodiment of the invention, the standardized medical gas delivery cord system comprises a unified design approach that ensures consistent compatibility and reliable operation across various medical gas delivery applications. The standardized medical gas delivery cord system, in one embodiment, integrates with a wearable device body that contains and manages the oxygen hose delivery system. This standardization extends to both the connection points and the overall cord configuration to maintain reliability across different usage scenarios.

In the preferred embodiment, the standardized system incorporates a dual-cord configuration. The first component comprises a stationary oxygen cord specifically designed for nose piece placement, while the second component features a fixed hose that connects to the retracting mechanism.

An embodiment of the invention includes standardized connection points that accommodate oxygen line lengths of up to 25 feet, with the system designed to maintain consistent gas flow and pressure throughout the delivery path. In another embodiment, the standardized system includes compatibility features that allow for the connection of additional line lengths beyond the standard 25 feet, enabling extended range capabilities of up to 50 feet while maintaining consistent delivery performance.

In accordance with an embodiment of the invention, the system incorporates an in-line connector port that enables the connection of additional oxygen line segments beyond the standard capacity contained within the wearable device housing. The in-line connector, in one embodiment, comprises a gas-tight coupling mechanism positioned at the terminal end of the oxygen line extending from the recoil mechanism, allowing for the attachment of supplementary line segments when extended range beyond the device's standard capacity is required. This modular connectivity approach enables patients to extend their effective range from the standard 25-foot capacity up to 50 feet or greater without requiring a complete device replacement.

In accordance with an embodiment of the invention, the extension coupling system maintains medical-grade gas-tight seals at all junction points through the use of standardized medical gas fittings compatible with ISO 5356-1 specifications. The coupling mechanism, in one embodiment, comprises threaded luer-lock connectors, push-to-connect quick-disconnect fittings, or compression-style barbed fittings that provide secure connections resistant to accidental disconnection during patient movement. The gas-tight seals ensure no oxygen leakage occurs at connection points, maintaining consistent oxygen delivery flow rates throughout the extended line length while preserving the safety characteristics of the visual indicator system.

In accordance with an embodiment of the invention, the additional line connection capability enables customization of the effective operating range to match specific patient needs or environmental constraints without compromising the automatic cord management functionality of the recoil mechanism. The base device manages the primary cord length through the automatic recoil mechanism, while the additional connected line segment extends outward from the device as a static extension, providing expanded range while maintaining the core benefits of automated cord management for the primary segment nearest the patient's body.

The preferred embodiment incorporates standardized safety features, including a visual indicator system with a red line positioned approximately one foot from the oxygen line's end, ensuring consistent safety protocols across all implementations of the system. In one embodiment, the standardized system is designed to work seamlessly with both circular and oblong disc housing configurations, maintaining compatibility regardless of the chosen form factor. The system's standardized design ensures reliable operation with the automatic recoil mechanism, allowing consistent performance during extension and retraction operations.

In a preferred embodiment of the invention, the standardized medical gas delivery cords are dimensioned and constructed using materials consistent with industry-standard oxygen delivery cords known in the art. The cords are specifically designed to maintain compatibility with existing oxygen delivery equipment and standard medical gas fittings. In one embodiment, the dual-cord system incorporates standard medical-grade tubing materials that ensure proper oxygen flow while maintaining flexibility and durability. The stationary oxygen cord for nose piece placement is configured to be compatible with standard nasal cannulas and other oxygen delivery accessories commonly used in medical settings.

The preferred embodiment features connection points that conform to industry-standard specifications, allowing for seamless integration with existing oxygen sources and delivery equipment. This standardization extends to both the fixed hose from the retracting mechanism and the stationary oxygen cord, ensuring universal compatibility across different medical gas delivery systems.

In an embodiment of the invention, the oxygen line components are dimensioned to maintain consistent gas flow and pressure throughout the standard length of up to 25 feet, as well as in extended configurations up to 50 feet. The materials and dimensions selected ensure that the automatic recoil mechanism can effectively manage the cord while maintaining proper gas flow characteristics.

In a preferred embodiment of the invention, the dual-cord system comprises two distinct components working in conjunction to provide efficient oxygen delivery. The first component consists of a stationary oxygen cord specifically designed for nose piece placement, while the second component features a fixed hose that integrates with the retracting mechanism.

In one embodiment, the stationary oxygen cord maintains a fixed position from the nose piece to the wearable device, ensuring stable and consistent oxygen delivery to the patient. This stationary configuration helps prevent disruption of oxygen flow during patient movement and minimizes the risk of disconnection.

In accordance with an embodiment of the invention, the dual-cord system provides a zero-tension facial interface wherein the stationary oxygen cord maintains less than 0.5 ounces of pulling force on the nasal cannula during full extension and retraction cycles of the recoil mechanism. This zero-tension characteristic, in one embodiment, is achieved through the isolation of the stationary oxygen cord from the mechanical forces of the automatic recoil mechanism, ensuring that spring retraction forces acting on the fixed hose component do not transmit through to the patient's nasal interface. The stationary cord experiences only passive tension resulting from its own weight and natural draping characteristics, not active pulling forces from spring-loaded retraction systems.

In accordance with an embodiment of the invention, the zero-tension facial interface eliminates mechanical tension transfer to the nasal interface during recoil operations, distinguishing the present invention from source-side recoil systems wherein spring forces pull directly on the oxygen delivery line connected to the patient's nose. In source-side configurations, retraction forces ranging from 2 to 8 ounces of linear pulling force act continuously on the oxygen line extending to the patient, creating constant pulling sensations at the nasal cannula interface and increasing the risk of cannula displacement or patient discomfort. The present invention's body-centric architecture eliminates this tension transfer by positioning the recoil mechanism on the patient's body and isolating the stationary oxygen cord from retraction forces.

In accordance with an embodiment of the invention, the zero-tension characteristic is maintained throughout the full range of patient movement including walking, bending, reaching, and transitional movements between standing and seated positions. The stationary oxygen cord's length is selected to provide sufficient slack for natural head movement and postural adjustments without introducing tension at the nasal interface, typically comprising a cord length of 2 to 4 feet between the wearable device at the patient's waist and the nasal cannula at the patient's face. This anthropometric dimensioning ensures the stationary cord never becomes taut during normal movement patterns, maintaining the zero-tension interface characteristic that enhances patient comfort and reduces cannula displacement risk.

The preferred embodiment incorporates a fixed hose component that connects directly to the retracting mechanism within the wearable device. This fixed hose is designed to work in harmony with the automatic recoil mechanism, enabling smooth extension when moving away from the oxygen source and controlled retraction when moving closer.

In an embodiment of the invention, both cords are configured to maintain consistent oxygen flow while supporting the system's extended range capabilities of up to 25 feet in standard configuration, with the ability to extend beyond this length when needed. The dual-cord design, in another embodiment, can accommodate configurations supporting up to 50 feet of flow while maintaining proper oxygen delivery performance.

The preferred embodiment features a dual-cord configuration that integrates seamlessly with the visual safety indicator system, incorporating the red line placement approximately one foot from the end of the oxygen line to prevent accidental disconnection. This design ensures that both the stationary cord and fixed hose work together to maintain proper oxygen delivery while enabling the automatic management of excess cord length.

In a preferred embodiment of the invention, the automatic recoil mechanism employs a spring-loaded design similar to that found in retractable dog leashes, providing automated management of oxygen line length during patient movement. The preferred embodiment features a dual-action capability, whereby the mechanism automatically retracts excess oxygen line when the patient moves toward the oxygen source, preventing tangling and tripping hazards. Conversely, when the patient moves away from the oxygen source, the mechanism smoothly extends the line to accommodate increased distance requirements.

In one embodiment, the recoil mechanism is housed within the wearable device body, integrating seamlessly with both circular and oblong disc configurations. The mechanism is designed to maintain consistent tension throughout the extension and retraction process, ensuring stable oxygen delivery while managing cord length.

The preferred embodiment thus incorporates a recoil system capable of managing standard oxygen line lengths of up to 25 feet. In another embodiment, the mechanism is scaled to accommodate extended lengths beyond 25 feet, with configurations supporting up to 50 feet of oxygen line while maintaining proper retraction and extension functionality.

In a preferred embodiment of the invention, the automatic recoil mechanism utilizes a spring-loaded spool assembly housed within the wearable device body. The mechanism comprises a central rotating drum or spool around which the oxygen line is wound, similar to configurations found in retractable dog leash mechanisms. The preferred embodiment incorporates a torsion spring assembly connected to the central spool, providing the necessary retraction force while maintaining consistent tension throughout the extension and retraction cycle. This spring assembly can be constructed of medical-grade stainless steel or other suitable materials that ensure durability and reliable operation.

In one embodiment, the recoil mechanism includes a ratchet and pawl system that works in conjunction with the torsion spring to provide controlled extension and retraction of the oxygen line. The ratchet mechanism allows for smooth extension when pulling the line away from the device while preventing unwanted retraction during use.

In accordance with an embodiment of the invention, the ratchet and pawl system incorporates tooth spacing and pawl engagement characteristics specifically calibrated to human gait patterns, optimizing the cord management response to natural walking movements. The ratchet teeth, in one embodiment, are spaced at intervals corresponding to the oxygen line length that deploys or retracts during a single walking stride, typically 0.5 to 1.5 inches of linear line movement per tooth engagement. This gait-synchronized tooth spacing ensures the ratchet engages and disengages at frequencies matching natural walking cadence, providing smooth cord management that responds fluidly to ambulation without introducing jerky or stuttering line movement.

In accordance with an embodiment of the invention, the pawl disengagement threshold is calibrated for typical walking stride length, wherein the directional change and momentum shift occurring at the end of each forward stride provides sufficient force variation to release the pawl from the ratchet teeth, allowing the torsion spring to retract accumulated slack. The pawl engagement force, typically calibrated to release at tension reductions of 0.5 to 1.0 ounces, ensures the mechanism responds to the subtle tension changes occurring during normal walking while maintaining engagement during stationary periods or slow transitional movements. This walking-optimized calibration distinguishes the device from generic retractable cord mechanisms designed for static extension and retraction rather than continuous dynamic movement.

In accordance with an embodiment of the invention, the one-foot safety distance provided by the red line visual indicator corresponds to exactly one step warning in human gait patterns, wherein the patient receives visual feedback precisely one stride before reaching maximum safe extension. The geometric relationship between ratchet tooth spacing, visual indicator placement, and human stride length creates an integrated system where the mechanical cord management rhythm synchronizes with natural walking patterns while the safety warning system provides temporally appropriate feedback aligned with the patient's movement cadence. This biomechanical synchronization enhances user experience and safety awareness compared to systems lacking gait-optimized mechanical characteristics.

The preferred embodiment features a housing assembly that contains the spool, spring, and ratchet components, designed to fit within either the circular or oblong disc and housing configurations of the wearable device. This housing can be constructed of lightweight, durable plastic materials consistent with the outer casing design.

In accordance with an embodiment of the invention, the recoil mechanism incorporates a hose spool comprising the central rotating drum component on which the oxygen hose is wound, distinctly separate from the hose housing which comprises the protective chamber surrounding the spool assembly. The hose spool, designated as reference number 304 in the figures, rotates about a central shaft during extension and retraction operations, with the oxygen hose winding onto or unwinding from the spool's cylindrical surface under control of the torsion spring assembly. The hose spool diameter, typically ranging from 2.5 to 5 inches depending on capacity requirements, is selected to maintain minimum bend radius specifications for the oxygen hose while maximizing capacity within the available housing volume.

In accordance with an embodiment of the invention, the hose housing comprises the stationary protective enclosure that contains the rotating hose spool, torsion spring assembly, ratchet mechanism, and associated components while shielding them from environmental exposure and physical contact. The hose housing, in one embodiment, comprises the inner and outer housing components that form the complete enclosure visible as the wearable device body. The hose housing provides structural support for the spool's bearing surfaces, maintains internal cleanliness to prevent debris interference with mechanical components, and presents smooth exterior surfaces compatible with patient contact during extended wear periods.

In accordance with an embodiment of the invention, the two-part architecture of hose spool and hose housing enables independent component optimization wherein the rotating spool can be engineered for rotational balance, capacity, and mechanical interface requirements while the surrounding housing can be optimized for protective functions, wearable ergonomics, and belt attachment features. This architectural separation facilitates serviceability, as the hose spool and wound oxygen hose can be removed as a complete assembly from the housing for replacement or capacity upgrade without disturbing the housing's belt attachment or exterior configuration.

In accordance with an embodiment of the invention, a spring cover comprises a protective housing element that encloses the torsion spring assembly, shielding the spring mechanism from exposure while ensuring hygienic operation throughout the device's service life. The spring cover, in one embodiment, comprises a molded plastic component that attaches to the inner housing and surrounds the torsion spring, preventing patient or caregiver contact with the spring's surface during housing opening for maintenance procedures. The spring cover eliminates the possibility of finger pinching or hair entanglement with the torsion spring during service operations while preventing dust accumulation or fluid contamination that could affect spring performance.

In accordance with an embodiment of the invention, the spring cover incorporates openings or pass-throughs that accommodate the spring's connection to the spool shaft and housing anchor points while maintaining protective enclosure of the spring's coiled body. The cover design, in one embodiment, allows visual inspection of spring condition through transparent material or inspection windows, enabling maintenance personnel to assess spring integrity without requiring cover removal. The protective function extends to preventing accidental spring unwinding or damage during spool removal procedures, as the cover constrains the spring even when the spool assembly is detached from its operational mounting position.

In accordance with an embodiment of the invention, the spring cover enhances operational safety by containing spring energy in the unlikely event of spring failure or disconnection, preventing the release of stored torsional energy that could cause spring components to separate violently. The cover's containment function provides an additional safety layer particularly valuable in medical device applications where patient safety considerations exceed those of consumer products. The hygienic operation ensured by the spring cover maintains the medical-grade cleanliness standards appropriate for devices in regular contact with patients receiving respiratory therapy.

In an embodiment of the invention, the recoil mechanism incorporates guide rollers or bushings that help manage the oxygen line during extension and retraction, ensuring smooth operation while preventing kinking or binding of the line. These components work together to maintain proper oxygen flow throughout the full range of motion, whether managing standard lengths up to 25 feet or extended configurations up to 50 feet.

In accordance with an embodiment of the invention, the kink prevention system comprises strategically positioned guide rollers and bushings that maintain minimum bend radius specifications throughout the oxygen line's path from the internal spool through the housing exit port. The guide roller assembly, in one embodiment, features medical-grade polymer rollers with smooth bearing surfaces that support the oxygen line during extension and retraction operations while preventing sharp bending that could occlude gas flow. The rollers are positioned at critical transition points where the oxygen line changes direction, including the spool tangent point, housing interior routing paths, and the exit port interface.

In accordance with an embodiment of the invention, an anti-kink collar is integrated at the housing exit point where the oxygen line transitions from the protected interior environment to external exposure. This collar comprises a flexible yet supportive sleeve constructed from medical-grade thermoplastic elastomer that maintains a minimum bend radius of 0.5 inches over a collar length of 1.5 to 2.0 inches. The anti-kink collar moves with the oxygen line during extension and retraction operations, providing continuous bend radius support throughout the dynamic range of motion while remaining flexible enough to accommodate the natural movement of the oxygen line during patient ambulation.

In accordance with an embodiment of the invention, the bushing components comprise low-friction guide surfaces positioned along the oxygen line's routing path to prevent line contact with sharp housing edges or mechanical components that could cause abrasion or kinking. These bushings, fabricated from materials such as polytetrafluoroethylene (PTFE) or ultra-high-molecular-weight polyethylene (UHMWPE), provide smooth sliding surfaces that minimize friction during line movement while protecting the oxygen line's outer jacket from wear. The combined action of guide rollers, anti-kink collar, and protective bushings ensures uninterrupted gas flow throughout the full extension range while extending oxygen line service life.

In a preferred embodiment of the invention, the automatic retraction functionality is achieved through the interaction between the torsion spring assembly and the central spool mechanism. As the patient moves toward the oxygen source, the stored energy in the torsion spring activates, causing the central spool to rotate and wind the oxygen line around the drum in a controlled manner. The retraction force is calibrated to provide sufficient tension for reliable cord management while avoiding excessive pull that could disturb the oxygen delivery or patient comfort.

In accordance with an embodiment of the invention, the torsion spring assembly incorporates a user-adjustable tension mechanism that enables customization of the retraction force to accommodate different patient strength levels, mobility characteristics, and comfort preferences. The tension adjustment mechanism, in one embodiment, comprises a rotatable collar or dial that modifies the preload on the torsion spring, increasing or decreasing the spring's effective force constant across a range typically spanning 2 to 8 ounces of linear retraction force at the oxygen line. This adjustability allows caregivers to calibrate the device for pediatric patients requiring minimal retraction force to avoid disturbing lightweight nasal cannulas, adult patients requiring moderate force for reliable cord management, or elderly patients with specific sensitivity requirements.

In accordance with an embodiment of the invention, the tension adjustment mechanism is accessible through a port or control located on the exterior housing surface, enabling adjustment without disassembling the device. The adjustment control, in one embodiment, comprises a recessed dial requiring a coin edge, pen tip, or specialized adjustment key to rotate, preventing accidental adjustment during normal handling while remaining accessible to caregivers for intentional modification. The dial features discrete detent positions corresponding to tension settings optimized for specific patient populations, with typical settings including: Position 1 (2-3 oz) for pediatric patients and sensitive applications; Position 2 (3-5 oz) for standard adult use; and Position 3 (5-8 oz) for applications requiring positive cord management in dynamic movement scenarios.

In accordance with an embodiment of the invention, visual or tactile indicators on the housing surface adjacent to the tension adjustment control identify the current tension setting, enabling caregivers to verify proper device configuration and maintain consistent settings across multiple devices in institutional care settings. The tension adjustment capability ensures optimal performance across the patient population spectrum while maintaining the safety characteristics of the recoil mechanism, as excessive retraction force could disturb oxygen delivery while insufficient force would fail to adequately manage cord slack and prevent tripping hazards.

In accordance with an embodiment of the invention, the torsion spring assembly is precisely calibrated to provide retraction force in the range of 2 to 5 ounces measured as linear pulling force at the oxygen line's free end. This calibrated force range, in one embodiment, is optimized to provide sufficient tension for reliable cord management and slack removal while remaining well below force thresholds that could disturb oxygen delivery or cause patient discomfort. The retraction force is specifically selected to be less than the weight of standard nasal cannula assemblies when accounting for friction and draping forces, ensuring the spring mechanism manages the oxygen line without transmitting noticeable pulling sensations to the patient's facial interface.

In accordance with an embodiment of the invention, the calibrated retraction force provides sufficient tension to overcome the oxygen line's natural stiffness and friction resistance during retraction operations, ensuring reliable slack removal across temperature ranges and throughout the oxygen line's service life. The force calibration accounts for friction generated at guide roller contact points, bushing surfaces, and housing exit ports, providing adequate margin to ensure complete retraction even when friction increases due to dust accumulation or material aging. The 2 to 5 ounce force range represents a carefully optimized balance wherein lower forces would fail to reliably retract line slack while higher forces would create unacceptable pulling sensations at the patient's nasal interface.

In accordance with an embodiment of the invention, the torsion spring assembly maintains constant force characteristics across the full extension range, providing consistent retraction tension whether the oxygen line is fully retracted, partially extended, or near maximum extension. This constant-force characteristic distinguishes the present invention from simple coil springs wherein force varies linearly with extension distance. The constant-force behavior, achieved through careful selection of torsion spring geometry and material properties, ensures predictable and reliable cord management throughout the device's operating range while preventing excessive force buildup at extended positions that could endanger the oxygen delivery connection.

The preferred embodiment incorporates a ratchet and pawl system that works in conjunction with the torsion spring to ensure smooth and controlled retraction. When the tension on the oxygen line decreases as the patient moves closer to the source, the pawl disengages from the ratchet teeth, allowing the spring-loaded spool to automatically rotate and retract the excess line. This automatic retraction process continues until either the desired amount of line is retrieved or the patient movement stops, at which point the pawl re-engages to maintain the current line length.

In accordance with an embodiment of the invention, the automatic recoil mechanism provides zero-latency mechanical response to patient movement, initiating cord extension or retraction within 0.1 seconds of movement initiation. This immediate mechanical response is achieved through the direct coupling between patient movement and the spring-loaded spool assembly, eliminating the sensor detection delays, signal processing times, and motor actuation latencies inherent in electronically-controlled cord management systems. The purely mechanical nature of the recoil mechanism ensures that cord deployment responds instantaneously to the patient's directional changes, providing continuous cord length optimization throughout dynamic movement patterns.

In accordance with an embodiment of the invention, the zero-latency response characteristic distinguishes the mechanical recoil system from motorized alternatives that require electronic sensors to detect patient position or movement, computerized controllers to interpret sensor data, and electric motors to actuate cord deployment. These electronic systems typically exhibit response latencies ranging from 0.5 to 2.0 seconds between movement initiation and cord adjustment, during which time excess slack accumulates or unwanted tension develops in the oxygen delivery cord. The present invention's mechanical system eliminates these delays through direct force coupling between the patient's movement and the torsion spring assembly, ensuring cord management actions occur simultaneously with patient movement rather than following a detection and response delay.

In accordance with an embodiment of the invention, a user-activated lock mechanism enables manual prevention of oxygen line retraction during specific activities where automatic recoil would be undesirable, such as sleeping, undergoing medical procedures, or during patient transfers. The lock mechanism, in one embodiment, comprises a sliding button, rotating collar, or lever positioned on the housing exterior that engages with the ratchet system to prevent pawl disengagement, effectively locking the spool in its current rotational position regardless of tension changes in the oxygen line. This manual locking capability supplements the automatic ratchet and pawl system's normal operation by providing override control when static line length is preferred.

In accordance with an embodiment of the invention, the lock mechanism operates through a sliding pin or blocking element that interferes with the pawl's normal disengagement motion, preventing the pawl from lifting out of the ratchet teeth even when tension decreases in the oxygen line. The lock control, in one embodiment, features a two-position design with "Auto" and "Lock" positions clearly marked on the housing surface, allowing patients or caregivers to quickly verify the current operational mode. In the "Auto" position, the blocking element retracts clear of the pawl mechanism, allowing normal automatic extension and retraction operations. In the "Lock" position, the blocking element prevents pawl disengagement, maintaining the current line extension length.

In accordance with an embodiment of the invention, the manual locking capability addresses specific use cases where automatic retraction could disturb patient comfort or interfere with clinical procedures. During sleeping periods, patients may prefer to lock the line at a comfortable extension length preventing the startle response that could result from unexpected line retraction. During medical procedures such as wound care, patient examinations, or imaging studies, caregivers may lock the line to maintain stable oxygen connection without automatic length adjustments that could interfere with positioning requirements. The lock mechanism enhances device versatility by providing both automatic and manual operating modes selectable based on immediate patient needs.

In an embodiment of the invention, the recoil mechanism works in conjunction with the visual safety indicator system, ensuring that the red line placement remains visible approximately one foot from the end of the oxygen line during both extension and retraction operations. This integration helps prevent accidental disconnection while allowing for automated cord management.

In a preferred embodiment of the invention, the visual safety indicator system comprises a distinctive red line marking (108) positioned approximately one foot from the end of the oxygen line, providing a critical disconnection prevention feature. The preferred embodiment incorporates this visual indicator directly onto the oxygen line surface, ensuring the marking remains visible during both extension and retraction operations of the automatic recoil mechanism. This placement allows users to quickly identify when the oxygen line is approaching its maximum safe extension limit.

In a preferred embodiment of the invention, the visual safety indicator system incorporates a permanent, high-visibility red line marking applied directly to the outer surface of the oxygen line using medical-grade, non-toxic pigments that maintain their visibility throughout the life of the device. The preferred embodiment positions the red line indicator precisely one foot from the terminal end of the oxygen line using automated measurement and marking processes to ensure consistent placement across all units. This standardized positioning provides healthcare workers and patients with a reliable visual reference point for monitoring safe operating distances.

In one embodiment, the red line marking extends circumferentially around the entire oxygen line to ensure visibility from any viewing angle during use. The marking width and contrast are optimized to be clearly visible under various lighting conditions commonly encountered in healthcare settings, including low-light environments. The preferred embodiment features a marking system that maintains its integrity during repeated extension and retraction cycles through the recoil mechanism. The indicator is designed to resist wear from regular contact with the guide rollers and internal components of the retracting mechanism while maintaining its high-visibility characteristics.

In accordance with an embodiment of the invention, the red line safety indicator extends completely around the oxygen line's circumference, forming a continuous band that ensures visibility from any viewing angle during use. The circumferential marking, in one embodiment, comprises a red-colored band with a width of 0.25 to 0.5 inches that extends 360 degrees around the oxygen line's exterior surface, eliminating blind spots that would exist with partial or single-sided markings. This omnidirectional visibility characteristic ensures caregivers can observe the safety indicator regardless of oxygen line orientation, which naturally varies due to twisting and rotation during patient movement.

In accordance with an embodiment of the invention, the 360-degree marking is applied using processes that ensure uniform color saturation and marking width around the entire circumference, preventing weak spots or gaps that could reduce visibility from certain viewing angles. Application methods, in one embodiment, comprise rotational spray coating, dip coating, heat-shrink sleeve application, or co-extrusion processes that inherently produce circumferential markings. The uniform circumferential coverage provides consistent visual conspicuity regardless of oxygen line rotation, which commonly occurs as the line passes through the recoil mechanism's guide rollers and exits the housing at varying rotational orientations depending on extension length and patient position.

In an embodiment of the invention, the visual indicator system includes supplementary markers or gradients leading up to the red line, providing additional visual feedback as the line approaches its disconnection point. These supplementary indicators work in conjunction with the primary red line to enhance safety awareness during patient movement and oxygen line management. The preferred embodiment ensures the visual indicator remains effective across the full range of supported configurations, from standard 25-foot lengths to extended 50-foot implementations, maintaining consistent visibility and warning functionality regardless of the selected operating length.

In accordance with an embodiment of the invention, the red line safety indicator incorporates photoluminescent pigments that absorb ambient light during normal illumination conditions and emit visible light in low-light or darkened environments. The photoluminescent material, in one embodiment, comprises strontium aluminate-based phosphors embedded within or applied to the red line marking, providing sustained visibility for periods of 4 to 8 hours following exposure to standard indoor lighting. This photoluminescent enhancement ensures the safety indicator remains visible during nighttime patient movement, in darkened patient rooms, or during power outages when artificial lighting may be unavailable.

In accordance with an embodiment of the invention, the red line marking incorporates reflective microbeads or retroreflective materials that enhance visibility under low-light conditions when even minimal ambient light sources are present. These reflective elements, comprising glass or ceramic microspheres with refractive indices optimized for maximum light return, are integrated into the red line marking during manufacturing. When illuminated by flashlights, nightlights, or other low-intensity light sources commonly present in healthcare environments, the reflective properties cause the red line to appear significantly brighter than non-reflective surfaces, ensuring the safety indicator can be readily identified even in challenging lighting conditions typical of nighttime caregiving scenarios.

In accordance with an embodiment of the invention, the supplementary gradient indicator system comprises a color-coded warning zone that provides progressive visual feedback as the oxygen line approaches maximum safe extension limits. The gradient system, in one embodiment, incorporates a yellow warning zone positioned at 2 to 3 feet from the line's terminal end, a transition zone featuring yellow-orange coloration at 1.5 to 2 feet from the end, and the primary red danger zone at the critical 0 to 1 foot final segment. This multi-stage visual feedback system provides early warning that allows patients and caregivers to anticipate approaching extension limits before entering the critical danger zone, enabling proactive movement adjustment rather than reactive response to the final red line indicator.

In accordance with an embodiment of the invention, tactile indicators comprising raised bumps, ridges, or texture changes are integrated along the oxygen line surface in coordination with the color gradient zones, providing safety feedback accessible to visually impaired patients or in low-light conditions where color discrimination may be compromised. The tactile indicators, in one embodiment, comprise small raised dots or continuous raised ribs molded into or adhered to the oxygen line surface, with tactile patterns differing between the yellow warning zone (single row of dots), transition zone (double row of dots), and red danger zone (continuous raised rib). The tactile system enables patients to assess extension status through touch when handling the oxygen line during connection, disconnection, or manual adjustment operations.

In accordance with an embodiment of the invention, the supplementary gradient indicators are coordinated with the automatic recoil mechanism's behavior, wherein the yellow warning zone emerges from the housing at extension levels of 70-85% of maximum capacity, providing advance notice before entering the critical final extension range. The progressive visual and tactile feedback system enhances safety awareness compared to single-point indicator systems, providing a graduated warning approach that accommodates patients with varying levels of visual acuity, color perception, and cognitive processing speed.

In one embodiment, the red line indicator works in conjunction with the dual-cord system, being implemented on both the stationary oxygen cord for nose piece placement and the fixed hose connected to the retracting mechanism. This configuration ensures consistent safety monitoring regardless of which portion of the system is being accessed or adjusted.

In accordance with an embodiment of the invention, the visual safety indicator system incorporates red line markings on both the stationary oxygen cord dedicated to nose piece placement and the fixed hose components extending from the retracting mechanism, providing redundant visual warnings regardless of which cord segment a caregiver or patient observes during operation. The dual-location marking strategy, in one embodiment, positions a red line indicator approximately one foot from the terminal connection point on the stationary oxygen cord and an additional red line indicator approximately one foot from the maximum extension point on the fixed hose, ensuring visual safety feedback is available at both critical disconnection risk zones within the dual-cord configuration.

In accordance with an embodiment of the invention, the dual-location indicator system provides enhanced safety awareness in clinical scenarios where caregivers may observe different portions of the oxygen delivery system depending on their position relative to the patient. A caregiver positioned near the patient's head naturally observes the stationary oxygen cord, while a caregiver positioned near the oxygen source or assisting with patient ambulation primarily observes the fixed hose from the retracting mechanism. By implementing red line indicators on both cord segments, the invention ensures visual safety feedback is available from multiple observation positions, reducing the risk that a caregiver might fail to observe disconnection risk due to limited visibility of a single-location indicator.

The preferred embodiment features a visual indicator that maintains its visibility across the system's full range of operation, from standard lengths of up to 25 feet to extended configurations supporting up to 50 feet of flow. The indicator's positioning approximately one foot from the line's end provides sufficient warning distance to prevent disconnection while allowing necessary movement and adjustment.

In an embodiment of the invention, the visual safety indicator is designed to be clearly visible in various healthcare environments and lighting conditions, helping to prevent accidental disconnection during patient movement or transfer situations. This safety feature integrates with both the circular and oblong disc housing configurations while maintaining its effectiveness during cord management operations.

In accordance with an embodiment of the invention, an audible safety alert system provides supplementary warning when the oxygen line approaches maximum safe extension limits, complementing the visual red line indicator system. The audible alert mechanism, in one embodiment, comprises a purely mechanical spring-actuated chime system that operates without electrical power or batteries, ensuring reliable operation throughout the device's service life. The alert mechanism activates when the red line marking enters a defined activation zone within the housing, triggering an audible click or tone that provides immediate feedback to the patient or caregiver.

In accordance with an embodiment of the invention, the mechanical chime mechanism comprises a spring-loaded striker element that engages with a resonating chamber when the oxygen line extends beyond a predetermined threshold corresponding to the red line safety zone. The striker element is positioned adjacent to the oxygen line's exit path and configured to contact the line when the red line indicator passes the exit port, converting the linear motion of the extending line into mechanical energy that drives the audible alert. The resonating chamber amplifies the striker impact to produce an audible tone in the 2000-4000 Hz frequency range, optimized for human hearing sensitivity while remaining non-startling to patients.

In accordance with an embodiment of the invention, the audible alert system provides multi-sensory feedback that enhances safety awareness for patients with visual impairments or in low-light conditions where the red line indicator may be difficult to observe. The mechanical nature of the alert system ensures consistent operation without maintenance requirements, battery replacement, or electronic failure modes. The audible feedback, in combination with the visual red line indicator, creates a redundant safety system that reduces the risk of accidental oxygen line disconnection during patient movement.

The preferred embodiment of the invention offers two distinct housing configurations to accommodate different user preferences and usage scenarios.

In one embodiment, the housing features a circular shape specifically designed to facilitate efficient coiled oxygen line storage. This circular configuration works in conjunction with the automatic recoil mechanism to maintain proper line organization and prevent tangling during storage and use.

The preferred embodiment also includes an alternative oblong disc configuration. This housing design provides a different form factor that may be more suitable for certain wearing positions or user preferences while maintaining full functionality of the recoil mechanism and line management system.

Both housing configurations are constructed using lightweight plastic materials to optimize portability while ensuring durability. The housing designs are specifically engineered to accommodate the internal components, including the recoil mechanism and standardized medical gas delivery system, while supporting oxygen line lengths up to 25 feet in standard configurations and up to 50 feet in extended range implementations.

In a preferred embodiment of the invention, the wearable device incorporates multiple attachment configurations to accommodate various user preferences and wearing styles.

The preferred embodiment features a belt strap connection system that allows for secure attachment directly to the user's belt. This belt strap configuration provides a removable yet stable mounting solution that can be easily adjusted or transferred as needed. In another embodiment, the device includes belt loop attachment points that enable the unit to be threaded onto a belt for more permanent placement. This configuration provides additional stability and security for users who prefer a more fixed mounting solution.

In accordance with an embodiment of the invention, the belt attachment system incorporates a quick-release snap mechanism that enables rapid attachment and detachment of the wearable device from the patient's belt without requiring threading the belt through fixed loops. The snap mechanism, in one embodiment, comprises a hinged clip assembly integrated into the rear surface of the wearable device housing that opens to accept the belt material and closes to secure the device in position. The clip assembly features a spring-loaded latch that maintains secure engagement during normal use while allowing intentional opening through manual pressure on a release button or lever.

In accordance with an embodiment of the invention, the quick-release mechanism provides critical safety benefits in emergency situations where rapid device removal may be necessary, such as during emergency medical procedures, imaging studies, or patient transfer operations requiring complete removal of all wearable items. The snap-on attachment allows caregivers to remove the device in under 3 seconds without requiring the patient to remove their belt or requiring manipulation of belt buckles or threading operations. This rapid-removal capability contrasts with thread-through belt loop designs that necessitate complete belt removal or time-consuming unthreading procedures.

In accordance with an embodiment of the invention, the snap-on clip mechanism incorporates sufficient gripping force to prevent inadvertent detachment during patient movement, typically providing retention forces of 5 to 10 pounds that exceed the forces encountered during normal ambulation while remaining below the threshold that would prevent intentional emergency removal. The clip jaws, in one embodiment, feature textured gripping surfaces or elastomeric inserts that enhance friction against belt materials ranging from leather to fabric to synthetic webbing, ensuring secure attachment across the variety of belt types commonly worn by patients. The tool-free attachment and detachment capability simplifies device donning and doffing procedures for patients with limited dexterity or cognitive impairments.

In accordance with the preferred embodiments described above, the present invention provides a comprehensive medical gas delivery system that addresses multiple challenges in oxygen delivery through an innovative combination of standardized components and automated management features.

The invention integrates a standardized medical gas delivery cord system with a wearable device body that can be secured through multiple attachment options. This integration, combined with the dual-cord system and automatic recoil mechanism, provides enhanced mobility and safety for oxygen therapy patients while effectively managing excess cord length.

The visual safety indicator system, featuring the distinctive red line placement, works in conjunction with the automatic recoil mechanism to prevent accidental disconnection during use. The lightweight plastic casing, available in both circular and oblong disc configurations, ensures optimal portability while maintaining system functionality.

Through these various embodiments, the invention successfully addresses the challenges of cord management, mobility limitations, and safety concerns commonly associated with traditional oxygen delivery systems. The system's extended range capabilities, supporting standard lengths up to 25 feet and configurations up to 50 feet, provide flexibility for various healthcare settings while maintaining consistent performance and reliability.

The combination of these features results in a comprehensive solution that enhances patient mobility, improves safety, and provides effective oxygen line management while maintaining compatibility with existing medical gas delivery equipment.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A wearable medical gas delivery system comprising:
a wearable device body configured to be worn at a patient's center of gravity via belt attachment;
a gas inlet port configured to receive pressurized medical gas from a stationary oxygen source;
a gas outlet port configured to deliver medical gas to a patient's respiratory interface;
an automatic recoil mechanism housed within said wearable device body, said automatic recoil mechanism comprising:
a gas-tight rotary manifold configured to maintain continuous gas flow during rotation, said gas-tight rotary manifold comprising a central stationary gas passage, a rotating gas outlet, and medical-grade seals maintaining gas-tight integrity across pressure ranges of 5 to 50 PSI;
a spring-loaded spool assembly operatively coupled to said gas-tight rotary manifold;
a torsion spring assembly providing calibrated retraction force in the range of 2 to 5 ounces; and
a ratchet and pawl system enabling controlled extension and automatic retraction;
a dual-cord configuration comprising:
a fixed hose operatively connected to said automatic recoil mechanism and extending from said gas inlet port, said fixed hose subject to retraction forces from said torsion spring assembly; and
a stationary oxygen cord extending from said gas outlet port to said patient's respiratory interface, said stationary oxygen cord mechanically isolated from said automatic recoil mechanism such that retraction forces are absorbed by said wearable device body and not transmitted to said patient's respiratory interface; and
a visual safety indicator system comprising a high-visibility colored marking positioned on said fixed hose approximately one foot from a terminal end, said marking comprising a circumferential band extending 360 degrees around said fixed hose;
wherein said wearable device body functions as a biomechanical tension buffer positioned at said patient's center of gravity, enabling omnidirectional patient movement while maintaining zero mechanical tension transfer to said patient's respiratory interface.

2. The wearable medical gas delivery system of claim 1, wherein said stationary oxygen cord maintains less than 0.5 ounces of pulling force on said patient's respiratory interface during full extension and retraction cycles of said automatic recoil mechanism.

3. The wearable medical gas delivery system of claim 1, wherein said gas-tight rotary manifold maintains consistent oxygen flow rates in the range of 1 to 6 liters per minute across a full extension range of 0 to 50 feet with pressure drop not exceeding 0.5 PSI.

4. The wearable medical gas delivery system of claim 1, wherein said automatic recoil mechanism provides zero-latency mechanical response to patient movement, initiating cord extension or retraction within 0.1 seconds of movement initiation through direct mechanical coupling without electronic sensors, battery power, or computerized controls.

5. The wearable medical gas delivery system of claim 1, wherein said ratchet and pawl system incorporates tooth spacing calibrated to human gait patterns, said ratchet teeth being spaced at intervals corresponding to oxygen line length that deploys or retracts during a single walking stride, and wherein said pawl disengagement threshold is calibrated such that directional changes occurring during walking provide sufficient force variation to release said pawl and allow said torsion spring to retract accumulated slack.

6. The wearable medical gas delivery system of claim 1, wherein said high-visibility colored marking comprises a red-colored band with a width of 0.25 to 0.5 inches, and wherein said visual safety indicator system further comprises a supplementary gradient indicator comprising a yellow warning zone positioned at 2 to 3 feet from said terminal end and tactile indicators providing safety feedback accessible to visually impaired patients.

7. The wearable medical gas delivery system of claim 1, wherein said wearable device body comprises a two-piece housing construction with a hinged connection enabling separation for internal access, and wherein said wearable device body is available in a circular housing configuration optimized for anterior belt placement and an oblong housing configuration optimized for lateral hip placement.

8. The wearable medical gas delivery system of claim 1, wherein said wearable device body comprises a contoured rear surface with a concave profile having a radius of curvature approximating a patient's waist or hip radius, said contoured rear surface distributing contact pressure during extended wear periods.

9. The wearable medical gas delivery system of claim 1, further comprising a quick-release snap mechanism integrated into a rear surface of said wearable device body, said snap mechanism enabling attachment and detachment from a patient's belt without requiring belt threading operations.

10. The wearable medical gas delivery system of claim 1, further comprising a user-activated lock mechanism positioned on an exterior of said wearable device body, said lock mechanism engaging with said ratchet system to prevent pawl disengagement and lock said spool in a current rotational position during sleeping, medical procedures, or patient transfers.

11. The wearable medical gas delivery system of claim 1, wherein said wearable device body comprises medical-grade materials conforming to ISO 10993 biocompatibility standards and selected for compatibility with healthcare disinfection protocols.

12. The wearable medical gas delivery system of claim 1, further comprising a kink prevention system comprising guide rollers positioned at critical transition points where said fixed hose changes direction and an anti-kink collar at a housing exit point maintaining a minimum bend radius of 0.5 inches.

13. The wearable medical gas delivery system of claim 1, wherein said spring-loaded spool assembly accommodates interchangeable spool configurations enabling a single housing design to support oxygen line lengths ranging from 25 feet to 50 feet through spool replacement.

14. A method for managing medical gas delivery tubing for a patient receiving supplemental oxygen therapy, said method comprising:
positioning a wearable device body at a patient's center of gravity via belt attachment;
receiving pressurized medical gas from a stationary oxygen source through a gas inlet port;
routing said pressurized medical gas through a gas-tight rotary manifold that maintains continuous gas flow during rotation of a spring-loaded spool assembly;
automatically retracting a fixed hose connected to said spring-loaded spool assembly when said patient moves toward said stationary oxygen source using a torsion spring assembly providing 2 to 5 ounces of retraction force;
automatically extending said fixed hose when said patient moves away from said stationary oxygen source;
delivering medical gas to said patient's respiratory interface through a stationary oxygen cord mechanically isolated from retraction forces generated by said torsion spring assembly;
absorbing all retraction forces at said wearable device body such that said patient's respiratory interface experiences less than 0.5 ounces of pulling force during extension and retraction operations; and
providing visual warning when said fixed hose approaches maximum safe extension by displaying a circumferential high-visibility colored marking positioned approximately one foot from a terminal end of said fixed hose.

15. The method of claim 14, wherein said automatic retracting and extending occur with zero-latency mechanical response within 0.1 seconds of patient movement initiation through direct mechanical coupling without electronic sensors or motors.

16. The method of claim 14, wherein said gas-tight rotary manifold maintains oxygen flow rates of 1 to 6 liters per minute across extension ranges of 0 to 50 feet with pressure drop not exceeding 0.5 PSI.

17. The method of claim 14, further comprising synchronizing cord management with human gait patterns through ratchet tooth spacing calibrated to oxygen line length deployed during a single walking stride.

18. A wearable medical gas delivery system comprising:
a biomechanical tension isolation hub configured to be worn at a patient's waist, said hub comprising:
a housing containing a gas-tight rotary manifold maintaining pressurized medical gas flow during continuous 360-degree rotation, a torsion spring assembly calibrated to provide 2 to 5 ounces of retraction force, and a ratchet and pawl system with tooth spacing synchronized to human gait patterns;
a gas inlet connecting to a stationary oxygen source;
a gas outlet connecting to a stationary oxygen cord leading to a patient's nasal cannula;
an automatic recoil mechanism managing a fixed hose extending from said hub to said stationary oxygen source; and
a circumferential visual safety indicator positioned one foot from a terminal end of said fixed hose;
wherein said hub employs a dual-cord architecture isolating said stationary oxygen cord from said automatic recoil mechanism such that spring retraction forces are absorbed at said hub and transferred to a patient's belt rather than said patient's nasal cannula, maintaining less than 0.5 ounces of pulling force at said nasal cannula during operation.

19. The wearable medical gas delivery system of claim 18, wherein said hub operates purely mechanically without electronic sensors, battery power, or computerized controls, providing zero-latency response within 0.1 seconds of patient movement initiation.

20. The wearable medical gas delivery system of claim 18, wherein said hub is positioned at said patient's center of gravity creating a body-centric management system that enables omnidirectional patient movement while maintaining zero mechanical tension transfer to said patient's nasal cannula throughout activities of daily living.
